(19) United States
(12) Patent Application Publication
(10) Pub. No.: US 2026/0191871 A1
(43) Pub. Date: Jul. 9, 2026

(54) USE OF ANTIVIRAL AGENTS, COMPOSITION OF MATTER, COMBINATION PREPARATIONS/AGENTS TO TREAT CHRONIC DISEASES ASSOCIATED WITH EPSTEIN-BARR VIRUS AND OTHER HUMAN HERPES VIRUSES

(71) Applicant: EPIPHANY BIOSCIENCES, San Francisco, CA (US)

(72) Inventor: Fred Volinsky, San Francisco, CA (US)

(21) Appl. No.: 19/133,094

(22) PCT Filed: Nov. 27, 2023
(86) PCT No.: PCT/US2023/081110

§ 371 Date: May 27, 2025

Related U.S. Application Data
(60) Provisional application No. 63/428,193, filed on Nov. 28, 2022.

(51) Int. Cl. A61K 31/522; A61K 9/00; A61K 9/08; A61K 9/20; A61K 9/48; A61K 31/357; A61K 31/506; A61K 31/513; A61K 31/519; A61K 31/675; A61K 38/21

(52) U.S. Cl. CPC A61K 31/522; A61K 9/0019; A61K 9/0053; A61K 9/08; A61K 9/20; A61K 9/48; A61K 31/357; A61K 31/506; A61K 31/513; A61K 31/519; A61K 31/675; A61K 38/215

(57) ABSTRACT

The present invention is directed to the use of antiviral agents, in particular valomaciclovir stearate and its polymorph Form A, as well as H2G (omaciclovir) to treat multiple sclerosis in combination with other agents, as well as the use of these agents to treat diseases and conditions such as chronic mononucleosis, long COVID, chronic fatigue syndrome, fibromyalgia, Crohn's disease, ulcerative colitis, rheumatoid arthritis, systemic lupus erythematosus, Graves' disease, Alzheimer's disease, mesial temporal lobe seizures, Epstein-Barr-virus-linked autism, or an Epstein-Barr-virus-linked cancer.

### CROSS-REFERENCES TO RELATED APPLICATIONS

This application claims the benefit of U.S. Provisional Patent Application Ser. No. 63/428,193 by Fred Volinsky, entitled “Use of Antiviral Agents, Composition of Matter, Combination Preparations/Agents to Treat Chronic Diseases Associated with Epstein-Barr Virus and Other Human Herpes Viruses,” and filed on Nov. 28, 2022, the contents of which are incorporated herein in their entirety in this application.

### FIELD OF THE INVENTION

This invention is directed to the use of polymorphs of valomaciclovir and the use of the antiviral agent H2G to treat acute and chronic diseases and cancers associated with Epstein-Barr virus and other herpesviruses, including multiple sclerosis, glioblastoma, acute infectious mononucleosis, and chronic mononucleosis, as well as complications and sequelae of infection by SARS-CoV2 such as long COVID.

### BACKGROUND OF THE INVENTION

Multiple sclerosis (MS) is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. Specific symptoms can include double vision, blindness in one eye, muscle weakness, and trouble with sensation or coordination. MS takes several forms, with new symptoms either occurring in isolated attacks (relapsing forms) or building up over time (progressive forms). In the relapsing forms of MS, between attacks, symptoms may disappear completely, although some permanent neurological problems often remain, especially as the disease advances.

While the cause of MS is unclear, one aspect of the pathophysiology is an autoimmune reaction that destroys myelin. There is increasing evidence that the autoimmune reaction can be triggered by a viral infection; genetic or environmental factors are also believed to play a role.

There is no known cure for multiple sclerosis. Treatments are basically palliative, including physical therapy and occupational therapy.

Multiple sclerosis is the most common immune-mediated disorder affecting the central nervous system. In 2022, it is estimated that there are nearly one million people in the United States with multiple sclerosis. In 2020, the worldwide estimate for occurrence of multiple sclerosis was about 2.8 million, with rates varying widely in different regions and among different populations. Typically, the onset of multiple sclerosis is between the ages of 20 and 50, and the disease is about twice as common in women as in men.

In recent years, there has been increasing evidence of the association of Epstein-Barr virus with multiple sclerosis and that infection with Epstein-Barr virus may in fact be a cause of multiple sclerosis (which does not necessarily rule out other causes of the disease) (C. V. Sumaya et al., “Epstein-Barr Antibodies in Multiple Sclerosis,” Arch. Neurol. 37: 94-96 (1980); P. F. Bray et al., “Epstein-Barr Virus Infection and Antibody Synthesis in Patients with Multiple Sclerosis,” Arch. Neurol. 40: 406-408 (1983); M. A. Moreno et al., “Molecular Signature of Epstein-Barr Infection in MS Brain Lesions,” Neurol. Neuroimmun. Neuroinflamm. 7: e466 (2018); A. Ascherio & K. L. Munger, “Environmental Risk Factors for Multiple Sclerosis, Part I: The Role of Infection,” Ann. Neurol. 61: 289-299 (2007); T. V. Lanz et al., “Clonally Expanded B Cells in Multiple Sclerosis Bind EBV EBNA1 and GlialCAM,” Nature 603: 321-327 (2022); W. H. Robinson & L. Steinman, “Epstein-Barr Virus and Multiple Sclerosis,” Science 375: 264-265 (2022); K. Bjornevik et al., “Longitudinal Analysis Reveals High Prevalence of Epstein-Barr Virus Associated with Multiple Sclerosis,” Science 375: 296-301 (2022)).

In addition to the association of Epstein-Barr virus with multiple sclerosis, there is increasing evidence for the association of Epstein-Barr virus and other viruses, particularly other herpesviruses, with other chronic diseases, particularly those affecting the central nervous system, such as long COVID, glioblastoma multiforme (GBM), and Alzheimer's disease (J. Rohrhofer et al., “Association Between Epstein-Barr-Virus Reactivation and Development of Long-COVID Fatigue,” Allergy 11:10.1111/all.15471 (2022); M. J. Peluso et al., “Impact of Pre-Existing Chronic Viral Infection and Reactivation on the Development of Long COVID,” medRxiv 22:2022.06.21.22276660 (2022); T. Chen et al., “Positive Epstein-Barr Virus Detection in Coronavirus Disease 2019 (COVID-19) Patients,” Sci. Rep. 11: 10902 (2021); S. Paolucci et al., “EBV DNA Increase in COVID-19 Patients with Impaired Lymphocyte Subpopulation Count,” J. Infect. Dis.: 104: 315-319 (2020); C. Soderberg-Naucler et al., “Survival in Patients with Glioblastoma Receiving Valganciclovir,” N. Eng. J. Med. 369: 985-986 (2013); G. Stragliotto et al., “Valganciclovir as Add-on to Standard Therapy in Glioblastoma Patients,” Clin. Cancer Res. 26: 4031-4039 (2020); C. Peng et al., “Valganciclovir and Bevacizumab for Recurrent Glioblastoma: A Single-Institution Experience,” Mol. Clin. Oncol. 4: 154-158 (2016); M. R. Pantalone et al., “Valganciclovir as Add-on to Second-Line Therapy in Patients with Recurrent Glioblastoma,” Cancers (Basel) 14: 1958 (2022); N. Z. Tzeng et al., “Anti-Herpetic Medications and Reduced Risk of Dementia in Patients with Herpes Simplex Virus Infections—A Nationwide, Population-Based Cohort Study in Taiwan,” Neurotherapeutics 15: 417-429 (2018); C. A. Mangold & M. L. Szpara, “Persistent Infection with Herpes Simplex Virus 1 and Alzheimer's Disease—A Call to Study How Variability in Both Virus and Host May Impact Disease,” Viruses 20: 966 (2019); R. D. Powell-Doherty et al., “Amyloid-β and p-Tau Anti-Threat Response to Herpes Simplex Virus 1 Infection in Primary Adult Murine Hippocampal Neurons,” J. Virol. 94: e1874-19 (2020); L. Letenneur et al., “Seropositivity to Herpes Simplex Virus Antibodies and Risk of Alzheimer's Disease: a Population-Based Cohort Study,” PLos One 3: e3637 (2008); H. Lövheim et al., “Reactivated Herpes Simplex Infection Increases the Risk of Alzheimer's Disease,” Alzheimers Dement. 11: 593-599 (2015); H. Lövheim et al., “Herpes Simplex Infection and the Risk of Alzheimer's Disease: A Nested Case-Control Study,” Alzheimers Dement. 11: 587-592 (2015); W A. Eimer et al., “Alzheimer's Disease-Associated β-Amyloid Is Rapidly Seeded by Herpesviridae to Protect Against Brain Infection,” Neuron 99: 56-63 (2018)).

In particular, with respect to glioblastoma multiforme, it was proposed that glioblastoma multiforme may be triggered by infection with cytomegalovirus, a human herpesvirus also designated as HHV-5. Based on this causal premise, valganciclovir was prescribed to a number of patients with glioblastoma multiforme. Subsequent data indicated that Epstein-Barr virus DNA was found in glioblastoma multiforme tumors but very little or no cytomegalovirus DNA. Cytomegalovirus is not a proven oncogenic virus, but Epstein-Barr virus is known to be oncogenic and has been linked with multiple cancers such as nasopharyngeal carcinoma.

Additionally, there have been a number of reports of herpesviruses being reactivated in response to immunosuppression or chemotherapy (S. Giordano et al., “Riattivazione di EBV in un Paziente Sottoposto a Chemioterapia per Metastasi da Timoma Misto Invasivo [EBV Reactivation in a Patient Undergoing Chemotherapy for Invasive Thymoma],” Infez. Med. 15: 195-198 (2007); I. Handous et al., “Co-Infections of Human Herpesviruses (CMV, HHV-6, HHV-7 and EBV) in Non-Transplant Acute Leukemia Patients Undergoing Chemotherapy,” Virol. J. 17:37 (2020)).

Although interferon has been described in combination with andrographolides (U.S. Pat. No. 9,060,994 to Orozco et al.), there is still a need for additional treatments, particularly for additional treatments that focus on the relationship between the reactivation of herpesvirus and the course of these diseases, including multiple sclerosis.

Therefore, there is a significant need for improved treatments for chronic diseases, particularly those affecting the central nervous system, such as, but not limited to multiple sclerosis, long COVID, glioblastoma multiforme, and Alzheimer's disease in which infection with a herpesvirus is associated with the pathophysiology of the chronic disease.

### SUMMARY OF THE INVENTION

One aspect of the present invention is a method for treating relapsing-remitting multiple sclerosis or HLA marker DR2 (DRB1*1501) progressive multiple sclerosis, the method comprising the steps of:
(1) administering H2G (omaciclovir) intravenously or intramuscularly at a dosage of about 5 mg/kg-25 mg/kg twice daily for 1 day to 6 weeks. The intramuscular injection may be given by a pre-loaded auto-injector so home administration is a possible option for administration. This auto-injector route of administration would apply to every use and utility mentioned in this patent for intramuscular administration of H2G (omaciclovir);
(2) subsequently administering high-dose valomaciclovir stearate for 1 week to 6 weeks at a dosage of about 1.5 g to about 2 g orally twice daily; and
(3) then subsequently administering chronic low-dose valomaciclovir stearate at a dosage of about 1 g to about 2 g once daily orally with chronic dosing. As an alternative patient treatment can be initiated with just oral therapy with valomaciclovir stearate without parenteral H2G treatment; in this alternative, treatment would start with high dose oral valomaciclovir stearate at about 1.5 g to about 2 g twice daily for 1 week to 6 weeks followed by low dose valomaciclovir stearate at about 1 g to 2 g orally once daily with chronic dosing.

Preferably, in this method and other methods according to the present invention, the valomaciclovir stearate is the Form A polymorph of valomaciclovir stearate as described below. Other polymorphic forms of valomaciclovir stearate exist.

Another aspect of the present invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks plus tenofovir at about 5 mg to about 150 mg in the H2G intravenous solution or given as a separate intravenous bolus or intramuscular injection for 1 day to 6 weeks; the tenofovir dose would depend on the patients renal function; in older patients or patients with impaired renal function the tenofovir dose would be in the lower range of about 10 mg to about 50 mg twice daily or withheld in case of true renal insufficiency; an alternative to intravenous or intramuscular tenofovir would be tenofovir disoproxil fumarate administered orally at about 10 mg to about 150 mg twice daily; dosing would depend on the renal state of a patient;
(2) subsequently administering high-dose valomaciclovir stearate at about 1.5 g to about 2 g twice daily orally combined with tenofovir disoproxil fumarate administered separately orally or in the form of a combination tablet or capsule containing both valomaciclovir stearate and tenofovir disoproxil fumarate; the daily dose of tenofovir is not to exceed 300 mg of tenofovir daily for 1 week to 6 weeks; the combination tablets or capsules would include 500 mg valomaciclovir stearate plus 50 mg to 75 mg tenofovir disoproxil fumarate. A lower dose of such a combination tablet for patients with less robust renal function would contain 500 mg-750 mg of valomaciclovir stearate plus 10 mg to 50 mg of tenofovir disoproxil fumarate tablets for oral administration twice daily for 1 to 6 weeks; in some alternatives, other agents can be added to the valomaciclovir stearate-tenofovir disoproxil fumarate combination tablets; such agents can include agents to improve blood-brain barrier penetration such as minoxidil or PDE₅-inhibitor agents such as sildenafil, or agents to increase gastrointestinal absorption such as pharmaceutical grade cod liver oil (in capsule form) which been reported to boost vitamin D levels and may have an anti-inflammatory effect which may both be beneficial in autoimmune diseases such as multiple sclerosis, or agents to prevent herpesvirus reactivation such as lysine; and
(3) subsequently administering a low dose valomaciclovir stearate therapy of about 1 g to 2 g daily oral chronic dosing. This chronic low dose of valomaciclovir stearate can be combined with tenofovir disoproxil fumarate at about 10 mg to 150 mg; lower doses of tenofovir disoproxil stearate would be recommended in patients with less robust renal function; tenofovir disoproxil fumarate would be withheld from patients with true renal impairment; this low dose valomaciclovir stearate chronic therapy or low dose chronic valomaciclovir/tenofovir disoproxil fumarate combination therapy or combination tablets or capsules could be combined with other agents such as agents to improve penetration of the blood-brain barrier, agents to improve gastrointestinal absorption, or agents to prevent reactivation of herpesvirus as described above. These agents could be prescribed separately or as a combination tablet or capsule, such as with cod liver oil.

Yet another aspect of the present invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks plus low-dose interferon type 1, for example Avonex (interferon beta-1a) once weekly intramuscularly at 7.5 μg once in week 1 and 15 μg once in week 2, and 30 μg once in weeks 3 and 4 or Betaseron (interferon beta-1 b) every other day subcutaneously with 0.0625 mg in week 1, 0.125 mg in week two; 0.1875 mg in week 3; and 0.25 mg in week 4;
(2) subsequently administering high-dose valomaciclovir stearate at about 1.5 g to about 2 g orally twice daily combined with low-dose interferon type 1 therapy as described in step (1); and
(3) then followed by low dose valomaciclovir stearate orally at about 1 g to 2 g once daily with chronic dosing.

In another alternative, therapy can alternatively begin with oral high dose therapy with valomaciclovir stearate and not be initiated with the intravenous or intramuscular H2G. In this case, therapy would begin with valomaciclovir stearate at 1.5 g to 2 g administered twice daily orally for 1 to 6 weeks followed by low dose valomaciclovir stearate administered once daily orally at about 1 g to about 2 g per day with chronic dosing.

Still another aspect of the invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks plus low-dose interferon type 1 twice daily as described above for interferon 1a and interferon-1b plus tenofovir intravenously or intramuscularly at 5 mg to 150 mg twice daily for 1 day to 6 weeks or tenofovir disoproxil fumarate at about 150 mg twice daily for 1 day to 6 weeks with lower doses for patients with less robust renal function as described above; and
(2) subsequently administering high-dose valomaciclovir stearate at about 1.5 g to 2 g twice daily combined with low-dose interferon type 1 (as described above) and low-dose tenofovir disoproxil fumarate orally once daily; the maximum daily dose of tenofovir disoproxil fumarate is 300 mg once daily.

Yet another aspect of the invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks or administering valomaciclovir stearate orally in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; and (iii) mycophenolate administered orally or intravenously at about 500 mg to about 1.5 mg twice daily for 1 day to 6 weeks; and
(2) subsequently administering chronic low-dose valomaciclovir stearate at a dosage of about 1 g to 2 g once daily.

In some alternatives according to the present invention, the method further comprises administration of a therapeutically effective quantity of an additional anti-integrin α4β1 agent. The anti-integrin α4β1 agent can be natalizumab. Natalizumab would be administered at 300 mg intravenously monthly.

In some alternatives according to the present invention, the method further comprises administration of a therapeutically effective quantity of an additional anti-CD20 agent. The anti-CD20 agent can be selected from the group consisting of rituximab, ocrelizumab, obinutuzumab, ofatumumab, ibritumomab tiuxetan, tositumomab, and ublituximab.

In some alternatives according to the present invention, the method further comprises administration of a therapeutically effective quantity of an additional anti-CD37 agent. The anti-CD37 agent can be selected from the group consisting of lilotomab satetrextan, Humalutin, naratuximab emtansine, and otlertuzumab.

Still another aspect of the invention is a method for preventing reactivation of Epstein-Barr virus comprising the step of administering a therapeutically effective quantity of valomaciclovir stearate in pill form together with oral lysine in a combination pill, wherein the quantity of oral lysine administered is about 200 mg to about 500 mg twice daily. The combination pill can further comprise a therapeutically effective quantity of H2G. The method can further comprise administering minoxidil at about 5 mg to about 40 mg orally twice daily, administering sildenafil at about 5 mg to about 20 mg orally twice daily, or administering vardenafil at about 5 mg to about 20 mg orally twice daily. As stated above, preferably, the valomaciclovir stearate is polymorph Form A of valomaciclovir stearate.

In some alternatives according to the present invention, valomaciclovir stearate polymorph A is administered orally in pill form together with an agent selected from the group consisting of minoxidil, sildenafil, and vardenafil.

In other alternatives according to the present invention, administration of intravenous H2G is followed by intranasal administration of a combination H2G/tenofovir spray twice daily.

Yet another aspect of the present invention is a method for treating a disease or condition selected from the group consisting of acute infectious mononucleosis, chronic mononucleosis, long COVID, chronic fatigue syndrome, fibromyalgia, Crohn's disease, ulcerative colitis, rheumatoid arthritis, SLE, Graves' disease, Alzheimer's disease, mesial temporal lobe seizures, Epstein-Barr-virus-linked autism, and an Epstein-Barr-virus-linked cancer, the method comprising the steps of:
(1) administering H2G intravenously or intramuscularly at about 5 mg/kg to about 25 mg/kg twice daily or administering valomaciclovir stearate polymorph Form A orally at a high dose of about 1.5 g to 2 g orally twice daily in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; and (iii) mycophenolate administered orally or intravenously at about 500 mg to about 1.5 mg twice daily for 1 day to 6 weeks;
(2) subsequently administering low-dose valomaciclovir stearate polymorph Form A at a dosage of about 1 g to 2 g once daily with chronic dosing; and
(3) then subsequently administering a therapeutically effective quantity of an additional agent selected from the group consisting of an anti-CD20 agent, an anti-integrin α4β1 agent, and an anti-CD37 agent.

In this method, the disease or condition can be an Epstein-Barr-virus-linked cancer, and the Epstein-Barr-virus-linked cancer can be selected from the group consisting of nasopharyngeal carcinoma, glioblastoma multiforme, and Kaposi's sarcoma.

Still another aspect of the present invention is a method for treatment of glioblastoma comprising the steps of:
(1) administering an induction agent selected from the group consisting of:
   (a) ribavirin administered at about 100 mg to about 400 mg twice daily for 1 day to 4 weeks;
   (b) romidepsin administered at about 14 mg/m² on days 1 and 8; and
   (c) sodium phenylbutyrate administered at a loading dose of about 250 mg intravenously over 90 minutes, followed by a dose of about 250 mg/kg per day for 7 days; followed by
(2) administering H2G intramuscularly or intravenously at a dose of about 5 mg/kg to about 25 mg/kg twice daily for 1 day to 6 weeks; followed by
(3) administering artesunate at a dose of about 1 mg/kg to about 2.4 mg/kg for 1 day to 6 weeks; followed by
(4) administering high dose polymorph Form A of valomaciclovir stearate at about 1.5 g to about 2 g twice daily for 1 week to 6 weeks, tenofovir disoproxil fumarate at about 75 mg twice daily for 1 day to 6 weeks, with the dose depending on the patient's renal function, and sildenafil at about 5 mg twice daily for 1 day to 6 weeks; and followed by
(5) administering about 850 mg of polymorph Form A of valomaciclovir stearate twice daily, tenofovir disoproxil fumarate at about 75 mg twice daily, with the dose and dosing duration depending on the patient's renal function with chronic dosing, and sildenafil at about 5 mg to about 20 mg twice daily with chronic dosing.

Yet another aspect of the invention is a formulation of polymorph Form A of valomaciclovir stearate for oral administration, the formulation comprising, per unit dose:
(1) 1000 mg of the Form A polymorph of valomaciclovir stearate;
(2) 180.53 mg of croscarmellose sodium;
(3) 42.31 mg of Povidone K-30;
(4) 13.08 mg of polysorbate 80;
(5) 53.21 mg of talc; and
(6) 9.60 mg of magnesium stearate;
   wherein the formulation is in the form of caplets.

Still another aspect of the present invention is a method for triple therapy of long COVID comprising the steps of:
(1) administering a therapeutically effective quantity of an induction agent for a human herpesvirus;
(2) administering a therapeutically effective quantity of at least one antiviral agent selected from the group consisting of H2G and polymorph Form A of valomaciclovir stearate, wherein the antiviral agent or agents are administered in two stages: a first high-dose stage and a second low-dose chronic stage; and
(3) administering a therapeutically effective quantity of an anti-B-cell agent selected from the group consisting of anti-CD20 agent, an anti-integrin α4β1 agent, or an anti-CD37 agent.

In this method, the induction agent can be selected from the group consisting of ribavirin, arginine, and an HDAC inhibitor; the HDAC inhibitor can be selected from the group consisting of valproic acid, sodium butyrate, romidepsin, abexinostat, belinostat, entinostat, givinostat, martinostat, mocetinostat, panobinostat, pracinostat, resminostat, sodium phenylbutyrate, tucidinostat, and vorinostat.

In this method, the antiviral therapy can be initiated at a high dose by administration of intravenous or intramuscular H2G (omaciclovir) at a dosage of about 5 mg/kg to about 25 mg/kg per day twice daily for 1 to 6 weeks. Alternatively, the antiviral therapy can be initiated at a high dose by administration of polymorph Form A of valomaciclovir stearate at about 1.5 g to 2 g orally twice daily, tenofovir disoproxil fumarate, and pharmaceutical grade cod liver oil at about 400 mg twice daily for 2 to 6 weeks.

In this method, the antiviral therapy can then be continued at a low dose by administration of polymorph Form A of valomaciclovir stearate at about 500 mg to about 850 mg, tenofovir disoproxil fumarate at about 75 mg, and pharmaceutical grade cod liver oil at about 80 mg to about 500 mg twice daily with chronic dosing. Alternatively, the antiviral therapy can be continued at a low dose by administration of polymorph Form A of valomaciclovir stearate at about 500 mg to about 850 mg, tenofovir disoproxil fumarate at about 75 mg, and sildenafil at about 5 mg to about 20 mg twice daily with chronic dosing.

Still another aspect of the invention is a capsule or tablet formulation comprising:
(1) polymorph Form A of valomaciclovir stearate at about 500 mg to about 850 mg per capsule or tablet;
(2) tenofovir disoproxil fumarate at about 75 mg per capsule or table (with daily dosing of tenofovir disoproxil fumarate dependent on the patient's renal status); and
(3) pharmaceutical grade cod liver oil at about 80 mg to about 500 mg per capsule or tablet.

Yet another aspect of the invention is a capsule or tablet formulation comprising:
(1) polymorph Form A of valomaciclovir stearate at about 500 mg to about 850 mg per capsule or tablet;
(2) tenofovir disoproxil fumarate at about 75 mg per capsule or tablet (with daily dosing of tenofovir disoproxil fumarate dependent on the patient's renal status); and
(3) sildenafil at about 5 mg to about 20 mg per capsule or tablet.

Still another aspect of the invention is a method for treating long COVID cases in which both persistent SARS-CoV-2 virus and reactivated Epstein-Barr virus or reactivated HHV6 virus is of concern, the method comprising the steps of:
(1) chronically administering a therapeutically effective quantity of nirmatrelvir;
(2) chronically administering a therapeutically effective quantity of ritonavir; and
(3) chronically administering a therapeutically effective quantity of polymorph Form A of valomaciclovir stearate.

In this method, in more detail, the method can comprise the steps of:
(1) chronic administration of about 75 mg of nirmatrelvir twice daily;
(2) chronic administration of about 50 mg of ritonavir twice daily; and
(3) chronic administration of about 750 mg of polymorph Form A of valomaciclovir stearate twice daily.

Yet another aspect of the present invention is a combination pill comprising:
(1) about 75 mg of nirmatrelvir per unit dose;
(2) about 50 mg of ritonavir per unit dose; and
(3) about 750 mg of polymorph Form A of valomaciclovir stearate per unit dose.

Methods as described above can further comprise administration of a therapeutically effective quantity of testosterone. Typically, the testosterone is administered by a method or device selected from the group consisting of injections, patches, buccal patches, implants, transdermal films, gels, and transdermal solutions.

Still another aspect of the present invention is a method of treating long COVID comprising the step of administering a therapeutically effective quantity of a combination of therapeutic agents selected from the group consisting of:
(a) valomaciclovir stearate and remdesivir, wherein the remdesivir is administered orally; and
(b) H2G and remdesivir, wherein the remdesivir is administered intravenously. Typically, the valomaciclovir stearate is the polymorphic Form A of valomaciclovir stearate.

In this method, when remdesivir and valomaciclovir stearate are administered, typically the active agents are administered in a dosage form that is pill, tablet, or capsule comprising about 300 mg valomaciclovir stearate plus about 700 mg remdesivir in each 1-gram tablet. In one alternative, the initial dose for long COVID is two 1-gram tablets twice daily for 4 days to 6 weeks followed by chronic dosing of one tablet once daily or twice daily. In another alternative, the dosage form is administered for 4 days to 6 weeks followed by once-daily dosing of either valomaciclovir stearate or a combination dosage form including valomaciclovir stearate, wherein the combination dosage form including valomaciclovir stearate is selected from: (i) a combination dosage form comprising a therapeutically effective quantity of valomaciclovir stearate and a therapeutically effective quantity of sildenafil; (ii) a combination dosage form comprising a therapeutically effective quantity of valomaciclovir stearate and a therapeutically effective quantity of tenofovir alafenamide fumarate; and (iii) a combination dosage form comprising a therapeutically effective quantity of valomaciclovir stearate and a therapeutically effective quantity of lysine.

Yet another aspect of the invention is a combination dosage form including valomaciclovir stearate, wherein the combination dosage form including valomaciclovir stearate is selected from: (i) a combination dosage form comprising a therapeutically effective quantity of valomaciclovir stearate and a therapeutically effective quantity of sildenafil; (ii) a combination dosage form comprising a therapeutically effective quantity of valomaciclovir stearate and a therapeutically effective quantity of tenofovir alafenamide fumarate; and (iii) a combination dosage form comprising a therapeutically effective quantity of valomaciclovir stearate and a therapeutically effective quantity of lysine. Typically, the valomaciclovir stearate is the polymorphic Form A of valomaciclovir stearate. The combination dosage form can be formulated as a pill, a capsule, a tablet, a liquid, or a nasal spray.

Still another aspect of the invention is a method of treating a disease or condition selected from the group consisting of long COVID, Alzheimer's disease, multiple sclerosis, chronic fatigue syndrome, fibromyalgia, acute infectious mononucleosis, and chronic mononucleosis comprising the step of administering a therapeutically effective quantity of valomaciclovir stearate and a therapeutically effective quantity of taurine. Typically, the valomaciclovir stearate is the polymorphic Form A of valomaciclovir stearate. Typically, the valomaciclovir stearate and taurine are administered in a capsule, pill, tablet, liquid, nasal spray, or patch.

Yet another aspect of the invention is a combination dosage form comprising a therapeutically effective quantity of valomaciclovir stearate and a therapeutically effective quantity of taurine. Typically, the valomaciclovir stearate is the polymorphic Form A of valomaciclovir stearate. Typically, the combination dosage form is formulated as a capsule, pill, tablet, liquid, nasal spray, or patch.

### DETAILED DESCRIPTION OF THE INVENTION

Other than in the operating examples, or where otherwise indicated, all numbers expressing quantities of ingredients, reaction conditions, and so forth used in the specification and claims are to be understood as being modified in all instances by the term “about.”

The following definitions are used in this application and apply unless specifically stated to the contrary:

Throughout this specification, the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

“Pharmaceutically acceptable salt” refers to a salt of valomaciclovir stearate or omaciclovir which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. As used herein, the term “valomaciclovir stearate” includes a pharmaceutically acceptable salt unless the use of such a pharmaceutically acceptable salt is expressly excluded.

The terms “subject,” “individual,” or “patient” are used interchangeably herein and refer to a vertebrate, preferably a mammal. Mammals include, but are not limited to, murines, rodents, simians, humans, farm animals, sport animals and pets. Unless specifically indicated to the contrary, compositions according to the present invention are not limited to use in humans, and methods according to the present invention are not limited to treatment of humans.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). Specific diseases and disorders that can be prevented by administration of compositions according to the present invention or by use of methods according to the present invention are described in further detail below.

The terms “treating,” “treatment,” or similar terminology, refer, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the subject. In yet other embodiments, “treating” or “treatment” refers to inhibiting the disease or disorder, either physically (e.g., stabilization of a discernible symptom), physiologically (e.g., stabilization of a physical parameter) or both. This can be demonstrated by, for example, but not by way of limitation, reduction in viral load, reduction of fever, reduction of pain, reduction of malaise, reduction of tissue destruction, or improvement in other clinical parameters, such as, in this context, reduction or stabilization of symptoms associated with neurological function or reduction or stabilization of cognitive impairment. In yet other embodiments, “treating” or “treatment” refers to delaying the onset of the disease or disorder. Use of the terms “treating,” “treatment,” or similar terminology is not to be taken as stating or implying a cure for any disease or condition.

“Compounds” refers to compounds encompassed by structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. Specific chemical structures are shown where appropriate and indicated by a specific formula designation with a Roman numeral, such as “Formula (I).”

The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures unless some alternatives are excluded.

Enantiomeric and stereoisomeric mixtures can be resolved into their component enantiomers or stereoisomers using separation techniques known in the art. Alternatively chiral synthesis techniques well known in the art can be used to synthesize specific enantiomers or diastereomers. The compounds may also exist in several tautomeric forms including the enol form, the keto form and mixtures thereof. Other forms of tautomerism, such as imine-enamine tautomerism, are also known in the art. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴N ¹⁵N, ¹⁷Q ¹⁸O, or other isotopes. Compounds may exist in unsolvated forms as well as solvated forms, including hydrated forms and as N-oxides. In general, compounds may be hydrated, solvated or N-oxides. Certain compounds may exist in multiple crystalline or amorphous forms or may exist in polymorphic forms.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, or other groups known in the art; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, and other groups known in the art. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

The term “alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, and other groups known in the art; butanyls such as butan-1-yl, butan-2-yl (sec-butyl), 2-methyl-propan-1-yl (isobutyl), 2-methyl-propan-2-yl (t-butyl), cyclobutan-1-yl, and other groups known in the art.

The term “alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s), leading to geometrical isomerism, unless a specific conformation is recited. Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, and other groups known in the art.

The term “alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, and other groups known in the art; butynyls such as but-1-yn-1-yl, but-1-yn-3-yl, but-3-yn-1-yl, and other groups known in the art.

The term “alkoxy,” by itself or as part of another substituent, refers to a radical of the formula —O—R^{a}, wherein R^{a} refers to alkyl as defined herein.

The term “alkyalkoxy,” by itself or as part of another substituent, refers to a radical of the form —R^{a}—O—R^{b}, wherein R^{a} and R^{b} each refer to alkyl as defined herein. The groups R^{a} and R^{b} may be the same or different.

The term “aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and other groups known in the art. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

The term “arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl. Other combinations of aryl and alkyl groups are known in the art.

The term “salts,” as used herein refers to salts of the compounds disclosed herein such as, for example, salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, propionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-napthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids. Hydrochloric acid salts are convenient in many applications and are generally well-tolerated pharmacologically.

The term “vehicle” as used herein refers to a diluent, adjuvant, excipient or carrier with which an antiviral agent or another therapeutic agent is administered.

As used herein, when a method refers to the administration of two or more compounds or classes of compounds, unless language such as “subsequently,” “followed by,” or equivalent terminology is employed, the two or more compounds or classes of compounds can be administered simultaneously or in any possible sequence. When language such as “subsequently,” “followed by,” or equivalent terminology is employed, the specified sequence of administration of the compounds or classes of compounds is intended.

### I. Antiviral Agents

Suitable antiviral agents for use in methods according to the present invention include valomaciclovir stearate and H2G (omaciclovir), as well as derivatives or analogs of these agents. Unless explicitly stated, recitation of valomaciclovir stearate or H2G (omaciclovir) include derivatives or analogs of these antiviral agents. However, use of valomaciclovir stearate or H2G (omaciclovir) is generally preferred in methods or compositions according to the present invention as described below with reference to those specific agents.

Valomaciclovir stearate has the systematic (IUPAC) name [(2R)-4-[(2S)-2-amino-3-methylbutanoyl]oxy-2-[(2-amino-6-oxo-3H-purin-9-yl)methyl]butyl]octadecanoate and the structure shown below in Formula (I):

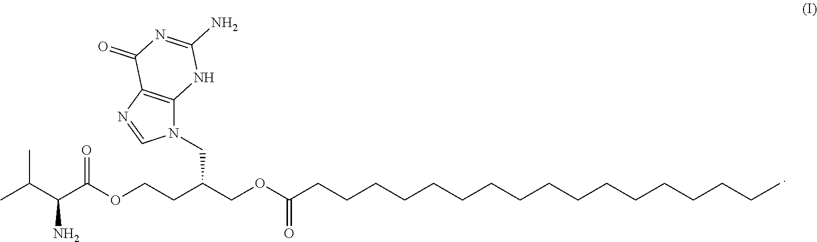 [SMILES: CCCCCCCCCCCCCCCCCC(=O)OC[C@H](CCOC(=O)[C@@H](N)C(C)C)Cn1cnc2c(=O)nc(N)[nH]c21]

Valomaciclovir stearate is the diester prodrug (valine and stearic acid) of the acyclic guanosine derivative H2G (omaciclovir) shown below in Formula (II)

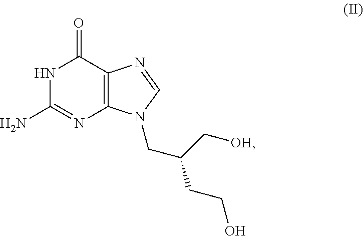 [SMILES: Nc1nc2c(ncn2C[C@H](CO)CCO)c(=O)[nH]1]

which has potent activity against human varicella zoster virus (VZV), Epstein-Barr virus (EBV), human herpesvirus-6 (HHV-6), HSV-1, and HSV-2. U.S. Pat. No. 5,869,493 to Engelhardt et al., incorporated herein by this reference, describes the preparation and activity of valomaciclovir stearate. The preparation and activity of valomaciclovir stearate has also been described in U.S. Pat. No. 7,189,849 to Leanna et al., incorporated herein by this reference, and in United States Patent Application Publication No. 2010/0240681 by O'Neill et al., incorporated herein by this reference.

Valomaciclovir stearate has been under development as an orally active agent against shingles (zoster) and other viral diseases. Valomaciclovir stearate was safe and well tolerated after multiple dosing with total daily doses of up to 6.0 g as shown in several phase I human clinical studies. Results from a phase II study (M98-829) using a suspension of valomaciclovir stearate at 250, 500, and 750 mg administered twice daily for 7 days to zoster patients, with acyclovir as a control, provided proof-of-concept for zoster lesion healing and a basis for further use of valomaciclovir stearate in patients with post-herpetic neuralgia.

Valomaciclovir stearate exists in a number of polymorphic forms, which are described in U.S. Pat. No. 9,492,456 to O'Neill et al. (“O'Neill et al. '456”) Of these polymorphic forms, a form designated as Polymorph A in O'Neill et al. '456 is the most crystalline and thermodynamically the most stable polymorph of valomaciclovir stearate. Methods for preparation of Polymorph A of valomaciclovir stearate are disclosed in O'Neill et al. '456. Other polymorphic forms of valomaciclovir stearate exist, including Form B1, Form B2, Form B3, Form C, and Form H. The Form A polymorph has 26 angles in X-ray diffraction of 22.9°±0.2°, 18.6°±0.2°, 19.5°±0.2°, 24.3°±0.2° 20.8°±0.2° 21.8±0.2°, 27.0±0.2° 14.7°±0.2°, 15.5°±0.2°, 25.5°±0.2°, and 29.9°±0.2°. Typically, the Form A polymorph has a purity of not less than 970 μg/mg on an anhydrous and solvent free basis. Typically, the Form A polymorph A has a potency of not less than 900 μg/mg on an anhydrous and solvent free basis.

Analogs of valomaciclovir stearate are disclosed in U.S. Pat. No. 5,869,493 to Engelhardt et al. (“Engelhardt et al. '493”) and include compounds of Formula (III):

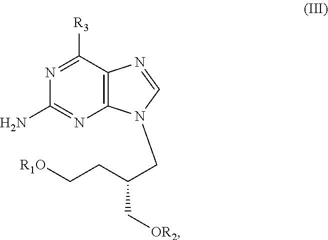

wherein:
(1) R₁ is —C(O)CH(CH(CH₃)₂)NH₂ or —C(O)CH(CH(CH₃)(CH₂CH₃)NH₂ and R₂ is —C(O)C₃-C₂₁ saturated or monounsaturated alkyl optionally substituted with up to five similar or different substituents independently selected from the group consisting of hydroxy, C₁-C₆ alkyl, C₁-C₆ alkoxy, C₁-C₆ alkoxy C₁-C₆ alkyl, C₁-C₆ alkanoyl, amino, halo, cyano, azido, oxo, mercapto, and nitro; or
(b) R₁ is —C(O)C₃-C₂₁ saturated or monounsaturated alkyl optionally substituted with up to five similar or different substituents independently selected from the group consisting of hydroxy, C₁-C₆ alkyl, C₁-C₆ alkoxy, C₁-C₆ alkoxy C₁-C₆ alkyl, C₁-C₆ alkanoyl, amino, halo, cyano, azido, oxo, mercapto, and nitro and R₂ is —C(O)CH(CH(CH₃)₂)NH₂ or —C(O)CH(CH(CH₃)(CH₂CH₃)NH₂; and
(c) R₃ is OH or H;
   or a pharmaceutically acceptable salt thereof.

These analogs include: (R)-9-[2-myristoyloxymethyl)-4-(L-valyloxy)butyl]guanine, (R)-9-[2-(oleoyloxymethyl)-4-(L-valyloxy)butyl]guanine, (R)-9-[2-(butyrloxymethyl)-4-(L-valyloxy)butyl]guanine, (R)-9-[2-(decanoyloxymethyl)-4-(L-valyloxy)butyl]guanine, (R)-9-[2-(docosanoyloxymethyl)-4-(L-valyloxy)butyl]guanine, (R)-9-[4-(L-isoleucyloxy)-2-(stearoyloxymethyl)butyl]guanine, (R)-9-[2-(decanoyloxymethyl)-4-(L-isoleucyloxy)butyl]guanine, (R)-9-[4-(L-isoleucyloxy)-2-(myristolyloxymethyl)butyl]guanine, (R)-9-[2-(4-acetylbutyryloxymethyl-4-(L-valyloxy)butyl]guanine, (R)-9-[2-dodecanoyloxymethyl-4-(L-valyloxy)butyl]guanine, (R)-9-[2-palmitoyloxymethyl-4-(L-valyloxy)butyl]guanine, (R)-2-amino-9-(2-stearoyloxymethyl-4-(L-valyloxy)butyl)purine, and (R)-9-[2-(L-valyloxymethyl)-4-(stearoyloxy)butyl]guanine.

Further analogs of valomaciclovir stearate are disclosed in U.S. Pat. No. 6,703,394 to Engelhardt et al. (“Engelhardt et al. '394”). These further analogs of valomaciclovir stearate are of Formula (IV):

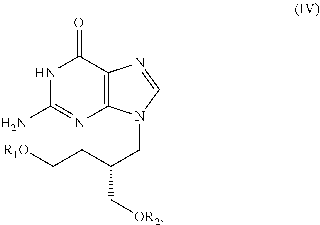

wherein:
(a) R₁ is —C(O)CH(CH(CH₃)₂)NH₂ or —C(O)CH(CH(CH₃)(CH₂CH₃)NH₂ and R₂ is —C(O)C₃-C₂₁ saturated or monounsaturated alkyl optionally substituted with up to five similar or different substituents independently selected from the group consisting of hydroxy, C₁-C₆ alkyl, C₁-C₆ alkoxy, C₁-C₆ alkoxy C₁-C₆ alkyl, C₁-C₆ alkanoyl, amino, halo, cyano, azido, oxo, mercapto, and nitro; or
(b) R₁ is —C(O)C₃-C₂₁ saturated or monounsaturated alkyl optionally substituted with up to five similar or different substituents independently selected from the group consisting of hydroxy, C₁-C₆ alkyl, C₁-C₆ alkoxy, C₁-C₆ alkoxy C₁-C₆ alkyl, C₁-C₆ alkanoyl, amino, halo, cyano, azido, oxo, mercapto, and nitro and R₂ is —C(O)CH(CH(CH₃)₂)NH₂ or —C(O)CH(CH(CH₃)(CH₂CH₃)NH₂; or a pharmaceutically acceptable salt thereof.

These analogs include: (R)-9-[2-(stearoyloxymethyl)-4-(L-valyloxy)butyl]guanine, (R)-9-[2-(myristoyloxymethyl)-4-(L-valyloxy)butyl]guanine, (R)-9-[2-(oleoyloxymethyl)-4-(L-valyloxy)butyl]guanine, (R)-9-[2-(butyrloxymethyl)-4-(L-valyloxy)butyl]guanine, (R)-9-[2-(decanoyloxymethyl)-4-(L-valyloxy)butyl]guanine, (R)-9-[2-(docosanoyloxymethyl)-4-(L-valyloxy)butyl]guanine, (R)-9-[4-(L-isoleucyloxy)-2-(stearoyloxymethyl)butyl]guanine, (R)-9-[2-(decanoyloxymethyl)-4-(L-isoleucyloxy)butyl]guanine, (R)-9-[4-(L-isoleucyloxy)-2-(myristoyloxymethyl)butyl]guanine, (R)-9-[2-(4-acetylbutyryloxymethyl-4-(L-valyloxy)butyl]guanine, (R)-9-[2-dodecanoyloxymethyl-4-(L-valyloxy)butyl]guanine, (R)-9-[2-palmitoyloxymethyl-4-(L-valyloxy)butyl]guanine, (R)-9-[2-(L-valyloxymethyl)-4-(stearoyloxy)butyl]guanine; or a pharmaceutically acceptable salt thereof.

Derivatives or analogs of H2G (omaciclovir) include monophosphate derivatives of H2G, diphosphate derivatives of H2G, and triphosphate derivatives of H2G. Derivatives or analogs of H2G further include a phosphate prodrug analog of H2G. Derivatives or analogs of H2G further include an analog of H2G selected from the group consisting of a compound of Formula (V):

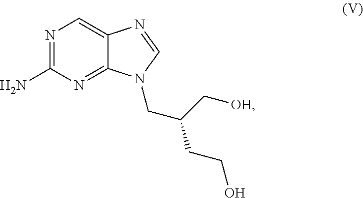 [SMILES: Nc1ncc2ncn(C[C@H](CO)CCO)c2n1]

and monophosphate derivatives, diphosphate derivatives, and triphosphate derivatives of analogs of H2G of Formula (V). Derivatives or analogs of H2G further include an analog of H2G selected from the group consisting of a compound of Formula (VI):

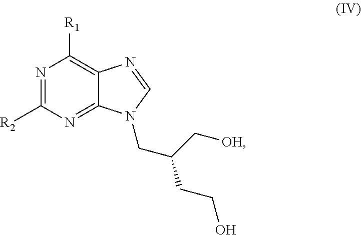

wherein:
(1) R₁ is hydrogen, hydroxy, mercapto, or amino; and
(2) R₂ is hydrogen, hydroxy, fluoro, chloro, or amino;
   and monophosphate derivatives, diphosphate derivatives, or triphosphate derivatives of analogs of Formula (VI); an analog of H2G selected from the group consisting of a compound of Formula (VII):

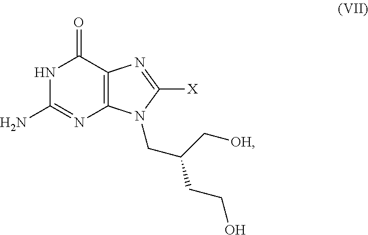

wherein X is selected from the group consisting of fluoro, chloro, bromo, iodo, —O-alkyl, and —S-alkyl, wherein the alkyl moieties are optionally substituted; and monophosphate derivatives, diphosphate derivatives, or triphosphate derivatives of analogs of Formula (VII); an analog of H2G selected from the group consisting of a compound of Formula (VIII):

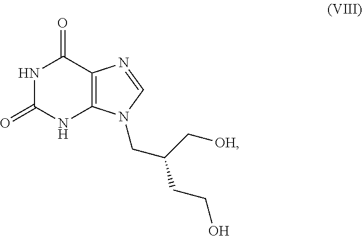 [SMILES: O=c1[nH]c(=O)c2ncn(C[C@H](CO)CCO)c2[nH]1]

and monophosphate derivatives, diphosphate derivatives, or triphosphate derivatives of analogs of Formula (VIII); an analog of H2G selected from the group consisting of a compound of Formula (IX):

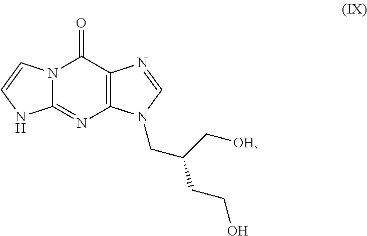 [SMILES: O=c1c2ncn(C[C@H](CO)CCO)c2nc2[nH]ccn12]

and monophosphate derivatives, diphosphate derivatives, or triphosphate derivatives of analogs of Formula (IX); an analog of H2G selected from the group consisting of a compound of Formula (X):

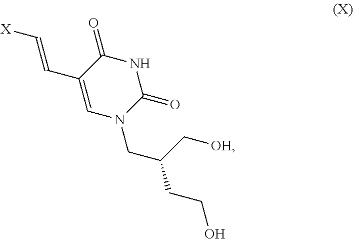

wherein X is selected from the group consisting of fluoro, chloro, bromo, iodo, —O-alkyl, and —S-alkyl, wherein the alkyl moieties are optionally substituted; and monophosphate derivatives, diphosphate derivatives, or triphosphate derivatives of analogs of Formula (X); an analog of H2G selected from the group consisting of a compound of Formula (XI):

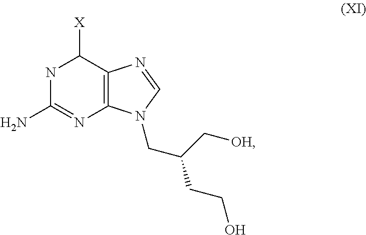

wherein X is selected from the group consisting of fluoro, chloro, bromo, iodo, —O-alkyl, and —S-alkyl, wherein the alkyl moieties are optionally substituted; and monophosphate derivatives, diphosphate derivatives, or triphosphate derivatives of analogs of Formula (XI); an analog of H2G selected from the group consisting of a compound of Formula (XII):

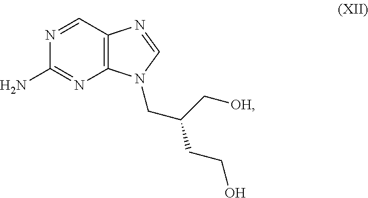 [SMILES: Nc1ncc2ncn(C[C@H](CO)CCO)c2n1]

and monophosphate derivatives, diphosphate derivatives, or triphosphate derivatives of analogs of Formula (XII); an analog of H2G selected from the group consisting of a compound of Formula (XIII):

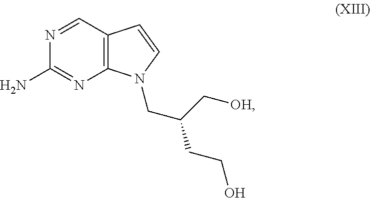 [SMILES: Nc1ncc2ccn(C[C@H](CO)CCO)c2n1]

and monophosphate derivatives, diphosphate derivatives, or triphosphate derivatives of analogs of Formula (XIII); an analog of H2G selected from the group consisting of a compound of Formula (XIV):

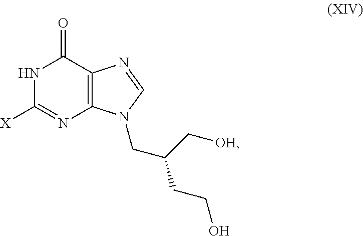

wherein X is selected from the group consisting of fluoro, chloro, bromo, iodo, —O-alkyl, and —S-alkyl, wherein the alkyl moieties are optionally substituted; and monophosphate derivatives, diphosphate derivatives, or triphosphate derivatives of analogs of Formula (XIV); an analog of H2G selected from the group consisting of a compound of Formula (XV):

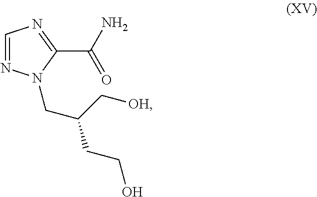 [SMILES: NC(=O)c1ncnn1C[C@H](CO)CCO]

and monophosphate derivatives, diphosphate derivatives, or triphosphate derivatives of analogs of Formula (XV); an analog of H2G selected from the group consisting of a compound of Formula (XVI):

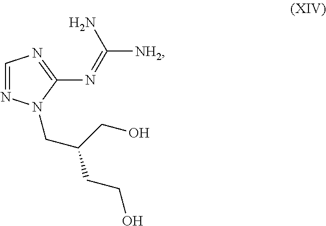 [SMILES: NC(N)=Nc1ncnn1C[C@H](CO)CCO]

and monophosphate derivatives, diphosphate derivatives, or triphosphate derivatives of analogs of Formula (XVI); an analog of H2G selected from the group consisting of a compound of Formula (XVII):

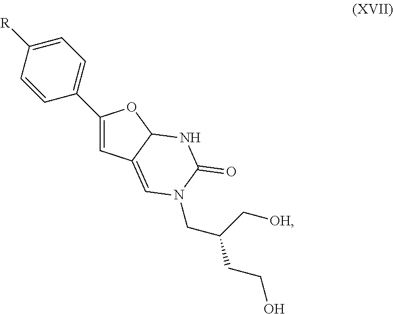

wherein R is selected from the group consisting of —(CH₂)ₙ—CH₃ wherein n is an integer from 0 to 11 and -(phenyl)-p-(CH₂)ₙ—CH₃ wherein n is an integer from 1 to 10; and monophosphate derivatives, diphosphate derivatives, or triphosphate derivatives of analogs of Formula (XVII); an analog of H2G selected from the group consisting of a compound of Formula (XVIII):

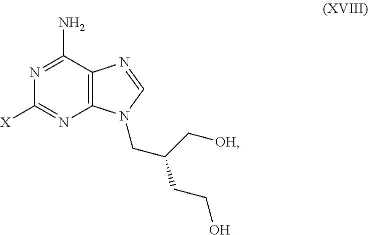

wherein X is selected from the group consisting of fluoro, chloro, bromo, iodo, —O-alkyl, and —S-alkyl, wherein the alkyl moieties are optionally substituted; and monophosphate derivatives, diphosphate derivatives, or triphosphate derivatives of analogs of Formula (XVIII); an analog of H2G selected from the group consisting of a compound of Formula (XIX):

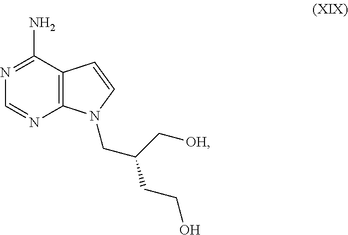 [SMILES: Nc1ncnc2c1ccn2C[C@H](CO)CCO]

and monophosphate derivatives, diphosphate derivatives, or triphosphate derivatives of analogs of Formula (XIX); an analog of H2G selected from the group consisting of a compound of Formula (XX).

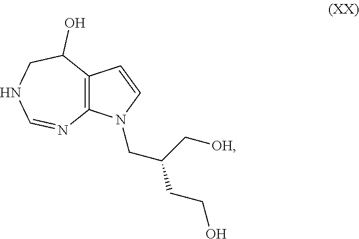 [SMILES: OCC[C@@H](CO)Cn1ccc2c1N=CNCC2O]

and monophosphate derivatives, diphosphate derivatives, or triphosphate derivatives of analogs of Formula (XVII); an ether or ester of a compound of Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), or Formula (XX); and an alkyl or arylalkyl derivative of a primary hydroxyl group of a compound of Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), Formula (XVII), Formula (XVIII), Formula (XIX), or Formula (XX).
II. Methods for Treating Diseases Affecting the Central Nervous System Associated with Herpesviral Infection with Antiviral Agents

With respect to the treatment of multiple sclerosis, latent Epstein-Barr virus (EBV) and the presence of the EBV nuclear antigen EBNA1 has a major causative role in MS. However, the peak incidence of MS is at ages 30-40 despite the fact that 75% of the United States population is seropositive for EBNA1 by age 18. If EBNA1 and latent EBV were the major driver of MS, it would be expected that the peak incidence age would be much closer to 18. Without being bound by this theory, Applicant believes that this disconnect between peak EBNA1 seroprevalence and peak MS incidence points to EBV reactivation and the presence of lytic EBV as the main trigger of EBV onset. In fact, if the presence of EBV1 and latent EBV were the main driver of MS and 95% of the United States population is seropositive for EBNA1 by age 18, one would therefore expect that the incidence of MS would be far higher. Without being bound by this theory, Applicant believes that four factors are involved in determining why only 300,000 Americans out of the 270 million seropositive Americans annually develop MS. These factors are EBV lytic reactivation, host genetics, vitamin D levels, and environmental factors such as yearly hours of sun exposure. Therefore, Applicant believes that lytic EBV, EBV load, and the prevention of reactivation may have a more prominent role in treating the autoimmune myelin-destroying process that plays a pathophysiological role in MS. The lytic EBV viral load may also serve as an amplification function analogous to a volume control on a stereo in terms of the quantitative amount of autoimmune cross-reactive antibodies that form, and, thus, suppressing EBV could slow or decrease disease progression; the higher the amount of EBV lytic activity, the more autoimmune activity is therefore expected to result. Suppressing EBV lytic activity should, therefore, decrease the amount of autoantibodies that cross-react with the myelin sheath, thereby decreasing the number of glial sclerae that develop on the white matter of the brain and spinal cord and thus slowing disease progression.

However, unlike other researchers in this field who have advocated antiviral agents to clear EBV from reservoirs to decrease the latent EBV load, Applicant advocates the use of anti-EBV agents to suppress lytic reactivation and lytic replication because Applicant believes that lytic replication, rather than latent EBV, may be a direct cause (not an indirect cause) of fatigue, cognitive loss, and depression in both progressive and relapsing-remitting MS. Without being bound by this theory, Applicant believes that suppressing lytic EBV should improve MS-associated depression and fatigue, and slow MS-associated cognitive loss which could be directly due to products of lytic EBV since similar symptoms are found in acute infectious mononucleosis which is characterized by lytic EBV replication.

Accordingly, one aspect of a method according to the present invention is a method for treating relapsing-remitting MS or HLA marker DR2 (formally termed DRB1*1501) progressive MS by administering H2G intravenously or intramuscularly at a dosage of about 5 mg/kg to about 25 mg/kg twice daily for 1 day to 6 weeks, followed by administering high-dose valomaciclovir stearate at 1.5 g to about 2 g orally twice daily for 1 week to 6 weeks followed by chronic low-dose valomaciclovir stearate at a dosage of about 1 g to 2 g once daily. Preferably, the valomaciclovir stearate is the Form A polymorph described above.

Another aspect of a method according to the present invention is a method for treating relapsing-remitting MS or HLA marker DR2 (formally termed DRB1*1501) progressive MS by combining administering H2G intravenously or intramuscularly at about 5 mg/kg to about 25 mg/kg twice daily for 1 day to 6 weeks plus tenofovir disoproxil fumarate orally at about 5 mg to about 150 mg twice daily or intravenously tenofovir at about 5 mg to about 150 mg daily for 1 day to 6 weeks (the dose depending on the patient's renal status), followed by administering high-dose valomaciclovir stearate at about 1.5 g to about 2 g orally twice daily for 1 week to 6 weeks combined with low-dose tenofovir disoproxil fumarate orally once daily. In another alternative of this method, the tenofovir disoproxil fumarate is replaced with interferon type 1, such as Avonex (interferon-1a) administered at a recommended dose range for Avonex once a week dose range 7.5 μg for first week titrated up to 30 μg per week to avoid influenza symptoms or initiate with 30 μg per week administered via an Avonex pre-filled syringe or an Avonex auto-injector intramuscularly or Betaseron (interferon-1b) administered subcutaneously. In yet another alternative of this method, the interferon type 1 is administered in addition to the tenofovir disoproxil fumarate. In both alternatives of these methods, the valomaciclovir stearate is preferably the Form A polymorph described above.

Yet another aspect of a method according to the present invention is a method of treating relapsing-remitting MS or HLA marker DR2 (DRB1*1501) progressive MS by administering H2G intravenously or intramuscularly at about 5 mg/kg to about 25 mg/kg BID for 1 day to 6 weeks or administering valomaciclovir stearate orally at a high dose of about 1.5 g to about 2 g orally twice daily for 1 week to 6 weeks in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; (iii) mycophenolate administered orally or intravenously at about 500 mg to about 1.5 mg twice daily for 1 day to 6 weeks and (iv) arginine administered orally at about 2 g to 3 g three times daily for 1 day up to 6 weeks; and subsequently administering chronic low-dose valomaciclovir stearate at a dosage of about 1 g to about 2 g once daily to chronically suppress EBV and decrease MS-associated fatigue, cognitive loss, and depression. In these alternatives of these methods, the valomaciclovir stearate is preferably the Form A polymorph described above.

In this alternative, typically, if ribavirin is administered, it is administered orally.

In another aspect of a method according to the present invention, the regimen described above, which employs high-dose H2G administered intravenously, followed by high-dose valomaciclovir stearate administered at about 1.5 g to 2 g orally twice daily for 1 week to 6 weeks, which is in turn followed by low-dose valomaciclovir stearate administered at about 1 g to about 2 g orally once daily for chronic use, and can also be combined with additional anti-integrin α4β1 agents such as natalizumab (Tysabri), which is an anti-integrin α4β1 monoclonal antibody, at a dosage of about 300 mg administered every 4 weeks, or with additional anti-CD20 agents such as monoclonal antibodies rituximab, ocrelizumab, obinutuzumab, ofatumumab, ibritumomab tiuxetan (in which the monoclonal antibody is conjugated to the radioisotope yttrium-90 or indium-111), tositumomab (which can be conjugated to the radioisotope iodine-131), or ublituximab, or with additional anti-CD37 agents, such as the monoclonal antibodies lilotomab satetrextan (Betalutin), Humalutin, naratuximab emtansine, and otlertuzumab.

In yet another aspect of a method according to the present invention, to prevent reactivation of Epstein-Barr virus, pills in which valomaciclovir stearate is the active ingredient or in which valomaciclovir stearate as an active ingredient is combined with one or more additional active ingredients (combination pills) that can be combined with oral lysine at a dosage of about 200 mg to about 500 mg for administration twice daily in a combination pill. The combination pill can be, in some alternatives, a triple combination pill including three active agents. The arginine/lysine ratio has an impact on herpesvirus reactivation or suppression.

To increase penetration of active agents through the blood-brain barrier, the H2G, administered intravenously, can be administered with minoxidil at about 5 mg to about 40 mg twice daily for 1 day to 6 weeks, sildenafil at about 5 mg to about 20 mg twice daily for 1 day to 6 weeks, or vardenafil at about 5 mg to about 20 mg twice daily for 1 day to 6 weeks. Oral valomaciclovir stearate can also be combined in a pill form with any of these agents to increase penetration of active agents through the blood-brain barrier.

In still another aspect of a method according to the present invention, administration of intravenous H2G as described above is followed by intranasal administration of a combination H2G/tenofovir spray twice daily.

In still another aspect of combination therapy according to the present invention, testosterone therapy may be of benefit in treating all forms of multiple sclerosis, long COVID, Alzheimer's disease, rheumatoid arthritis, and chronic fatigue syndrome. All these diseases have been linked in the literature to the reactivation of HHVs such as EBV, HHV6a, HSV1 and herpes zoster. Also, all these disease have a common gender predominance; about 70%-80% of patients with these diseases are females and about 20% males. If this is the case then could the testosterone/estrogen ratio have an impact on treating all these diseases? Could testosterone levels have an impact on the cell-mediated immune system and low testosterone levels could result in HHV reactivation and reactivation of viruses involved in the listed diseases. Testosterone therapy is proposed as adjunct therapy to all these human herpesvirus-linked diseases listed in this application. It is proposed that in addition to valomaciclovir, H2G and all the combinations and methods listed within this application, testosterone therapy should be utilized as an additional option. Testosterone injections, patches, or buccal patches are recommended, and can be used to administer the following strengths: 30 mg/1.5 mL; 2.5 mg/24 hr; 5 mg/24 hours; cypionate 100 mg/mL; cypionate 200 mg/mL; 2 mg/24 hr; 4 mg/24 hr; 25 mg/2.5 g (1%); 50 mg/5 g (1%); 40.5 mg/2.5 g (1.62%); 20.25 mg/1.25 g (1.62%); 20.25 mg/actuation (1.62%); 12.5 mg/1.25 g (1%); enanthate 200 mg/mL; 100 mg/mL; propionate 100 mg/mL; enanthate 100 mg/mL; 50 mg/mL; micronized; propionate; 10 mg/0.5 g; 75 mg; 6 mg/24 hr; propionate micronized; cypionate; 5.5 mg/0.122 g; 30 mg/12 hr; undecanoate 112.5 mg; enanthate 100 mg/0.5 mL; enanthate 50 mg/0.5 mL; enanthate 75 mg/0.5 mL; 1%; 2%; undecanoate 250 mg/mL; 158 mg; 198 mg; 237 mg. Intramuscular injection can be utilized with testosterone undecanoate: 25 mg-750 mg (3 mL) intramuscular injection followed by 25-750 mg (3 mL) injected after 4 weeks, then 25-750 mg (3 mL) every 10 weeks thereafter. Testosterone enanthate and cypionate can be administered at 20 mg to 400 mg by injection every 2 to 4 weeks. Implants can alternatively be used with 2 to 6 pellets (75 mg each) implanted subcutaneously every 3 to 6 months. The number of pellets to be implanted depends upon the minimal daily requirements of testosterone propionate administered parenterally. Thus, two 75 mg pellets are implanted for each 25 mg testosterone propionate required weekly. The chronological and skeletal ages must be taken into consideration, both in determining the initial dose and in adjusting the dose during treatment. This drug should be used only if the benefits outweigh the serious risks of pulmonary oil microembolism and anaphylaxis. Injections more frequently than once every two weeks are not recommended. Adequate effect of the implants (pellets) continues or three to four months, sometimes as long as six months A mucoadhesive oral patch can be used by applying a 30 mg patch to the gum region twice a day; morning and evening (about 12 hours apart). A transdermal film can be used with 2 to 6 mg applied to the back, abdomen, upper arm, or upper thigh once a day, preferably at night. Gel (in tubes, packets or spray) can be used with 5 g applied once a day, preferably in the morning. Consult the manufacturer product information for specific dosage and additional instructions for use. A transdermal solution can alternatively be used; the initial dose is 60 mg of testosterone (1 pump actuation of 30 mg of testosterone to each axilla), applied once a day, at the same time each morning. Consult the manufacturer product information for specific dosage and additional instructions of use.

Yet another aspect of a method according to the present invention employs a triple therapy approach to treating relapsing-remitting MS and progressive HLA DR2 MS, using: (i) an induction agent to induce transient lytic reactivation of Epstein-Barr virus; (ii) one or more antiviral agents targeting Epstein-Barr virus; and (iii) an anti-CD20 agent, anti-integrin α4β1 agent, or anti-CD37 agent. Specifically, the induction agent can be ribavirin, romidepsin (administered intravenously), sodium phenylbutyrate, or arginine as described above. The antiviral agent targeting Epstein-Barr virus can be: (i) H2G administered intravenously or intramuscularly at about 5 mg/kg to about 25 mg/kg twice daily 1 day for 6 weeks; (ii) oral high-dose valomaciclovir stearate administered at about 1.5 g to 2 g twice daily orally for 1 week to 6 weeks, followed by low-dose valomaciclovir stearate administered at about 1 g to about 2 g orally once daily for chronic use; or (iii) beginning therapy with just oral high dose valomaciclovir stearate plus induction agents listed in this patent plus anti CD20 agents, anti-integrin α4β1 agents or anti-CD 37 agents; oral high-dose valomaciclovir stearate can be administered at about 1.5 g to about 2 g orally twice daily with any of the induction agents listed in this patent such as arginine, ribavirin, intravenous romidepsin, the anti-CD20 agent, anti-integrin α4β1 agent, or anti-CD37 agent as described above.

This triple therapy regimen as described, employing administration of an induction agent, followed by administration of one or more antiviral agents, and then subsequently followed by administration of an additional agent selected from the group selected from an anti-CD20 agent, an anti-integrin α4β1 agent, and an anti-CD37 agent, can be used for the treatment of additional diseases and conditions, including, but not limited to, chronic mononucleosis, long COVID, chronic fatigue syndrome, fibromyalgia, Crohn's disease, ulcerative colitis, rheumatoid arthritis, systemic lupus erythematosus, Graves' disease, Alzheimer's disease, mesial temporal lobe seizures, Epstein-Barr-virus-linked autism, and Epstein-Barr-virus-linked cancers including, but not limited to, nasopharyngeal carcinoma, glioblastoma multiforme, and Kaposi's sarcoma.

In particular, with respect to glioblastoma multiforme, Applicant proposes, without being bound by this theory, that a more potent antiviral agent, such as H2G or valomaciclovir stearate coupled with induction agents and coupled with additional agents to potentiate the antiviral effect of the H2G or the valomaciclovir stearate, such as oral artesunate, mycophenolate, tenofovir disoproxil fumarate, or ribavirin, plus administration of an anti-CD20 agent, anti-integrin α4β1 agent, or anti-CD37 agent as described above, will have a far greater impact on impeding glioblastoma multiforme tumor progression and on survival time for glioblastoma multiforme patients.

In one method of treatment of glioblastoma multiforme according to the present invention, induction is performed with ribavirin administered at about 100 mg to about 400 mg twice daily for 7 days or with romidepsin administered at about 14 mg/m² on days 1 and 8 or with sodium phenylbutyrate administered at a loading dose of about 250 mg intravenously over 90 minutes, followed by a dose of about 250 mg/kg per day for 7 days, then followed by administration of H2G at a dose of about 5 mg/kg to about 25 mg/kg twice daily for 1 day to 6 weeks, followed by administration of artesunate at a dose of about 1 mg/kg to about 2.4 mg/kg for 1 day to 6 weeks, followed by administration of polymorph Form A of valomaciclovir stearate at about 1.5 g to 2 g twice daily orally (administered as two tablets) for 1 week to 6 weeks, tenofovir disoproxil fumarate at about 75 mg twice daily for 1 week to 6 weeks (depending on the renal status of the patient), and sildenafil at about 5 mg twice daily for 1 day to 6 weeks, then followed by administration of about 850 mg of polymorph Form A of valomaciclovir stearate twice daily, tenofovir disoproxil fumarate at about 5 mg to about 75 mg twice daily for two to four weeks, and sildenafil at about 5 mg to 20 mg twice daily chronically.

Formulations for administration of H2G and valomaciclovir stearate can be determined by one of ordinary skill in the art and depend on factors such as the intended dosage, the route of administration, the frequency of administration, the duration of administration, and the physical form of the composition to be administered.

In some alternatives according to the present invention, the formulation for H2G and Valomaciclovir Stearate Polymorph A can include a delivery agent compound selected from the group consisting of N-(8-[2-hydroxybenzoyl]-amino)caprylic acid, N-(10-[2-hydroxybenzoyl]-amino)decanoic acid, 8-(2-hydroxy-4-methoxybenzoylamino)octanoic acid, 8-(2,6-dihydroxybenzoylamino)octanoic acid, 8-(2-hydroxy-5-bromobenzoylamino)octanoic acid, 8-(2-hydroxy-5-chlorobenzoylamino)octanoic acid, 8-(2-hydroxy-5-iodobenzoylamino)octanoic acid, 8-(2-hydroxy-5-methylbenzoylamino)octanoic acid, 8-(2-hydroxy-5-fluorobenzoylamino)octanoic acid, 8-(2-hydroxy-5-methoxybenzoylamino)octanoic acid, 8-(3-hydroxyphenoxy)octanoic acid, 8-(4-hydroxyphenoxy)octanoic acid, 6-(2-cyanophenoxy)hexanoic acid, 8-(2-hydroxyphenoxy)octyl-diethanolamine, 8-(4-hydroxyphenoxy)octanoate, 8-(4-hydroxyphenoxy)octanoate, 8-(2-hydroxy-4-methoxybenzoylamino)octanoic acid, 8-(2-hydroxy-5-methoxybenzoylamino)octanoic acid, and salts thereof. Preferred salts include, but are not limited to, monosodium and disodium salts.

In some alternatives according to the present invention, the formulation for H2G and valomaciclovir stearate can include an excipient selected from the group consisting of a preservative, a sweetening agent, a thickening agent, a buffer, a liquid carrier, an isotonic agent, a wetting, solubilizing, or emulsifying agent, an acidifying agent, an antioxidant, an alkalinizing agent, a carrying agent, a chelating agent, a colorant, a complexing agent, a solvent, a suspending and or viscosity-increasing agent, a flavor or perfume, an oil, a penetration enhancer, a polymer, a stiffening agent, a protein, a carbohydrate, a bulking agent, and a lubricating agent. Suitable alternatives for such excipients can be determined by one of ordinary skill in the art based on the factors described above. Pharmaceutically acceptable excipients may be added to facilitate manufacture, enhance stability, control release, enhance product characteristics, enhance bioavailability, drug absorption or solubility, optimize other pharmacokinetic considerations, optimize the pharmaceutical formulation for a route of administration, enhance patient acceptability, or for another reason related to manufacture, storage, or use of a pharmaceutical composition. Excipients used in pharmaceutical compositions according to the present invention are compatible with the pharmaceutically active agent or agents included in the pharmaceutical composition, are compatible with other excipients included in the pharmaceutical composition, and are not injurious to and are tolerated by any patients to whom the pharmaceutical composition is administered.

As is generally known in the art of pharmaceutical formulation, a particular excipient can fulfill one or more of these functions in a particular pharmaceutical composition, depending on the concentration of the excipient, the other excipients in the composition, the physical form of the composition, the concentration of active agent in the composition, the intended route of administration of the composition, and other factors. The recitation of a particular excipient in a category below is not intended to exclude the possible use of the excipient in another category or categories.

Typically, the liquid carrier can be, but is not limited to, a liquid carrier selected from the group consisting of saline, phosphate buffered saline, glycerol, and ethanol.

Typically, the isotonic agent can be, but is not limited to, a polyalcohol selected from the group consisting of mannitol and sorbitol, sodium chloride, and potassium chloride.

Typically, the wetting or emulsifying agent is a surfactant. Typically, the surfactant is selected from the group consisting of benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol, acacia, cholesterol, diethanolamine, glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, triethanolamine, emulsifying wax, cetomacrogol, and cetyl alcohol.

Typically, the preservative is selected from the group consisting of benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, diazolidinyl urea, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, and thymol.

Typically, the buffer is selected from the group consisting of acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, sodium bicarbonate, Tris (Tris(hydroxymethyl)aminomethane), MOPS (3-(N-morpholino)propanesulfonic acid), HEPES (N-(2-hydroxyethyl)piperazine-N′-(2-ethanesulfonic acid), ACES (2-[(2-amino-2-oxoethyl)amino]ethanesulfonic acid), ADA (N-(2-acetamido)2-iminodiacetic acid), AMPSO (3-[(1,1-dimethyl-2-hydroxyethylamino]-2-propanesulfonic acid), BES (N,N-bis(2-hydroxyethyl)-2-aminoethanesulfonic acid, Bicine (N,N-bis(2-hydroxyethylglycine), Bis-Tris (bis-(2-hydroxyethyl)imino-tris(hydroxymethyl)methane, CAPS (3-(cyclohexylamino)-1-propanesulfonic acid), CAPSO (3-(cyclohexylamino)-2-hydroxy-1-propanesulfonic acid), CHES (2-(N-cyclohexylamino)ethanesulfonic acid), DIPSO (3-[N,N-bis(2-hydroxyethylamino]-2-hydroxy-propanesulfonic acid), HEPPS (N-(2-hydroxyethylpiperazine)-N′-(3-propanesulfonic acid), HEPPSO (N-(2-hydroxyethyl)piperazine-N′-(2-hydroxypropanesulfonic acid), MES (2-(N-morpholino)ethanesulfonic acid), triethanolamine, imidazole, glycine, ethanolamine, phosphate, MOPSO (3-(N-morpholino)-2-hydroxypropanesulfonic acid), PIPES (piperazine-N,N′-bis(2-ethanesulfonic acid), POPSO (piperazine-N,N′-bis(2-hydroxypropaneulfonic acid), TAPS (N-tris[hydroxymethyl)methyl-3-aminopropanesulfonic acid), TAPSO (3-[N-tris(hydroxymethyl)methylamino]-2-hydroxy-propanesulfonic acid), TES (N-tris(hydroxymethyl)methyl-2-aminoethanesulfonic acid), tricine (N-tris(hydroxymethyl)-methylglycine), 2-amino-2-methyl-1,3-propanediol, and 2-amino-2-methyl-1-propanol.

Typically, the acidifying agent is selected from the group consisting of acetic acid, citric acid, fumaric acid, hydrochloric acid, diluted hydrochloric acid, malic acid, nitric acid, phosphoric acid, diluted phosphoric acid, sulfuric acid, and tartaric acid.

Typically, the antioxidant is selected from the group consisting of ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, sodium thiosulfate, sulfur dioxide, and tocopherol.

Typically, the alkalinizing agent is selected from the group consisting of strong ammonia solution, ammonium carbonate, diethanolamine, diisopropanolamine, potassium hydroxide, sodium bicarbonate, sodium borate, sodium carbonate, sodium hydroxide, and trolamine.

Typically, the carrying agent is selected from the group consisting of acacia syrup, aromatic syrup, aromatic elixir, cherry syrup, cocoa syrup, orange syrup, syrup, corn oil, mineral oil, peanut oil, sesame oil, bacteriostatic sodium chloride for injection and bacteriostatic water for injection.

Typically, the chelating agent is selected from the group consisting of edetate disodium, ethylenediaminetetraacetic acid, citric acid, and salicylates.

Typically, the coloring agent is selected from the group consisting of ferric oxides red, yellow, black or blends, FD&C Red No. 3, FD&C Red No. 20, FD&C Yellow No. 6, FD&C Blue No. 2, D&C Green No. 5, D&C Orange No. 5, D&C Red No. 8, and dyes suitable for pharmaceutical use.

Typically, the complexing agent is selected from the group consisting of ethylenediaminetetraacetic acid, salts of ethylenediaminetetraacetic acid, gentisic acid ethanolamide, and oxyquinoline sulfate.

Typically, the solvent is selected from the group consisting of acetone, ethanol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl isobutyl ketone, mineral oil, oleic acid, peanut oil, polyethylene glycol, propylene carbonate, propylene glycol, sesame oil, water for injection, sterile water for injection, sterile water for irrigation, and purified water.

Typically, the suspending and/or viscosity-increasing agent is selected from the group consisting of acacia, agar, alginic acid, aluminum monostearate, bentonite, purified bentonite, magma bentonite, carbomers, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, Veegum, and xanthan gum.

Typically, the flavor or perfume is selected from the group consisting of anise oil, cinnamon oil, menthol, anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, and vanillin.

Typically, the oil is selected from the group consisting of arachis oil, mineral oil, olive oil, sesame oil, cottonseed oil, safflower oil, corn oil, and soybean oil.

Typically, the penetration enhancer is selected from the group consisting of monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones, and ureas.

Typically, the polymer is selected from the group consisting of cellulose acetate, alkyl celluloses, hydroxyalkylcelluloses, acrylic polymers and copolymers, polyesters, polycarbonates, and polyanhydrides.

Typically, the stiffening agent is selected from the group consisting of hydrogenated castor oil, cetostearyl alcohol, cetyl alcohol, cetyl esters wax, hard fat, paraffin, polyethylene excipient, stearyl alcohol, emulsifying wax, white wax, and yellow wax.

Typically, the sweetening agent is selected from the group consisting of aspartame, dextrates, dextrose, excipient dextrose, fructose, glycerol, mannitol, propylene glycol, saccharin, calcium saccharin, sodium saccharin, sorbitol, solution sorbitol, sucrose, compressible sugar, confectioner's sugar, and syrup.

Typically, the protein is selected from the group consisting of bovine serum albumin, human serum albumin (HSA), recombinant human albumin (rHA), gelatin, and casein.

Typically, the carbohydrate is selected from the group consisting of fructose, maltose, galactose, glucose, D-mannose, sorbose, lactose, sucrose, trehalose, cellobiose, raffinose, melezitose, maltodextrins, dextrans, starches, mannitol, maltitol, lactitol, xylitol, sorbitol, and myoinositol.

Typically, the bulking agent is selected from the group consisting of polypeptides and amino acids.

Typically, the lubricating agent is selected from the group consisting of magnesium stearate, stearic acid, sodium lauryl sulfate, and talc.

Pharmaceutical compositions such as described above can be formulated for oral, sustained-release oral, buccal, sublingual, inhalation, insufflation, or parenteral administration. Suitable routes for administration of pharmaceutical compositions according to the present invention can be chosen based on factors known to one of skill in the art including the unit dose of the active agent or agents or the derivative, analog, salt, solvate, or prodrug of such active agent or agents and, if present, the other active agent or agents, the particular carriers or excipients included in the composition, the intended route of administration, the disease or condition to be treated, its severity, other diseases or conditions affecting the and other factors known in the art.

If a pharmaceutical composition according to the present invention is intended for oral administration, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms. The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

In one alternative, a sustained-release formulation is used. Sustained-release formulations are well-known in the art. For example, they can include the use of polysaccharides such as xanthan gum and locust bean gum in conjunction with carriers such as dimethylsiloxane, silicic acid, a mixture of mannans and galactans, xanthans, and micronized seaweed, as disclosed in U.S. Pat. No. 6,039,980 to Baichwal. Other sustained-release formulations incorporate a biodegradable polymer, such as the lactic acid-glycolic acid polymer disclosed in U.S. Pat. No. 6,740,634 to Saikawa et al. Still other sustained-release formulations incorporate an expandable lattice that includes a polymer based on polyvinyl alcohol and polyethylene glycol, as disclosed in U.S. Pat. No. 4,428,926 to Keith. Still other sustained-release formulations are based on the Eudragit™ polymers of Rohm & Haas that include copolymers of acrylate and methacrylates with quaternary ammonium groups as functional groups as well as ethylacrylate methylmethacrylate copolymers with a neutral ester group.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (e.g., mannitol) as appropriate.

When compositions according to the present invention are formulated for parenteral administration, e.g., formulated for injection via the intravenous, intramuscular, subcutaneous, intralesional, or intraperitoneal routes or other routes known in the art, many options are possible. The preparation of an aqueous composition as described above will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified. The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

For administration of the active agent or active agents such as, but not limited to, H2G and valomaciclovir stearate described in the present invention or of a pharmaceutical composition containing the active agent or agents described in the present invention, various factors must be taken into account in setting suitable dosages. These factors include other medications being administered to the subject, which, in some cases, may alter the pharmacokinetics of the agent or agents being administered, either causing it to be degraded more rapidly or more slowly. These medications can, for example, affect either liver or kidney function or may induce the synthesis of one or more cytochrome P450 enzymes that can metabolize the active agent or agents.

The exact formulation, route of administration and dosage can be chosen by the individual physician in view of the patient's condition. (See, e.g., Fingl et al., in The Pharmacological Basis of Therapeutics, 1975, ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated, such as, but not limited to, a malignancy, and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

For oral administration, active agents as described above that are suitable for oral administration are typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms.

The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical preparations for oral use can be obtained by combining the active compound or compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses or different doses of a single active compound.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycol. In addition, stabilizers may be added.

In some alternatives, a sustained-release formulation is used. Sustained-release formulations are well-known in the art. For example, they can include the use of polysaccharides such as xanthan gum and locust bean gum in conjunction with carriers such as dimethylsiloxane, silicic acid, a mixture of mannans and galactans, xanthans, and micronized seaweed, as disclosed in U.S. Pat. No. 6,039,980 to Baichwal. Other sustained-release formulations incorporate a biodegradable polymer, such as the lactic acid-glycolic acid polymer disclosed in U.S. Pat. No. 6,740,634 to Saikawa et al. Still other sustained-release formulations incorporate an expandable lattice that includes a polymer based on polyvinyl alcohol and polyethylene glycol, as disclosed in U.S. Pat. No. 4,428,926 to Keith. Still other sustained-release formulations are based on the Eudragit™ polymers of Rohm & Haas that include copolymers of acrylate and methacrylates with quaternary ammonium groups as functional groups as well as ethylacrylate methylmethacrylate copolymers with a neutral ester group.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (e.g., mannitol) as appropriate.

One skilled in the art recognizes that the route of administration is an important determinant of the rate of efficiency of absorption for any particular therapeutically active compound. For example, the alimentary route, e.g., oral, rectal, sublingual, or buccal, is generally considered the safest route of administration. The delivery of the drugs into the circulation is slow, thus eliminating rapid high blood levels of the drugs that could potentially have adverse acute effects. Although this is considered the safest route of administration, there are several disadvantages. One important disadvantage is that the rate of absorption varies, which is a significant problem if a small range in blood levels separates a drug's desired therapeutic effect from its toxic effect, i.e., if the drug has a relatively low therapeutic index. Also, patient compliance is not always ensured, especially if the rectal route of administration is chosen or if oral administration is perceived by the patient as unpleasant. Furthermore, with oral administration, extensive hepatic metabolism can occur before the drug reaches its target site. Another route of administration is parenteral, which bypasses the alimentary tract. One important advantage of parenteral administration is that the time for the drug to reach its target site is decreased, resulting in a rapid response, which is essential in an emergency. Furthermore, parenteral administration allows for delivery of a more accurate dose. Parenteral administration also allows for more rapid absorption of the drug, which can result in increased adverse effects. Unlike alimentary administration, parenteral administration requires a sterile formulation of the drug and aseptic techniques are essential. The most significant disadvantage to parenteral administration is that it is not suitable for insoluble substances. In addition to alimentary and parenteral administration routes, topical and inhalation administrations can be useful. Topical administration of a drug is useful for treatment of local conditions; however, there is usually little systemic absorption. Inhalation of a drug provides rapid access to the circulation and is the common route of administration for gaseous and volatile drugs, or drugs that can be vaporized or nebulized. It is also a desired route of administration when the targets for the drug are present in the pulmonary system, which is the case for compositions and methods according to the present invention.

In some alternatives according to the present invention, administration of one or more active agents is by inhalation. Typically, the administration of an active agent by inhalation comprises administration of a dose administered by use of a pressurized metered dose inhaler (pMDI), dry powder inhaler, or nebulizer; the administration of the dose by inhalation may or may not generate measurable blood levels of nadolol in the range associated by oral dosing. Typically, the inhaled dose will be delivered by pMDI in the range of from about 1% to about 10% of the minimally effective oral dose.

A pressurized metered dose inhaler consists of three major components; the canister which is produced in aluminum or stainless steel by means of deep drawing, where the formulation resides; the metering valve, which allows a metered quantity of the formulation to be dispensed with each actuation; and an actuator (or mouthpiece) which allows the patient to operate the device and directs the aerosol into the patient's lungs. The formulation itself is made up of the drug, a liquefied gas propellant and, in many cases, stabilizing excipients. The actuator contains the mating discharge nozzle and generally includes a dust cap to prevent contamination. To use the inhaler the patient presses down on the top of the canister, with their thumb supporting the lower portion of the actuator. Actuation of the device releases a single metered dose of the formulation which contains the medication either dissolved or suspended in the propellant. Breakup of the volatile propellant into droplets, followed by rapid evaporation of these droplets, results in the generation of an aerosol consisting of micrometer-sized medication particles that are then inhaled. Pressurized metered dose inhalers are disclosed in U.S. Pat. No. 10,806,701 to Bonelli et al. Further details about metered dose inhalers are provided in P. B. Myrdal et al., “Advances in Metered Dose Inhaler Technology: Formulation Development,” AAPS Pharm. Sci. Tech. 15: 434-455 (2014), which discloses alternatives for formulations suitable to be administered by metered dose inhalers.

Dry powder inhalers commonly hold the medication either in a capsule for manual loading or in a proprietary form inside the inhaler. Once the inhaler is loaded or actuated, the operator inserts the mouthpiece of the inhaler into his or her mouth and takes a sharp, deep inhalation, ensuring that the medication reaches the lower parts of the lungs, holding his or her breath for 5-10 seconds. Some powder inhalers use lactose as an excipient. Dry powder inhalers are disclosed in U.S. Pat. No. 10,842,952 to Bilgic.

Nebulizers use oxygen, compressed air, or ultrasonic power to break up solutions or suspensions into small aerosol droplets that are inhaled from the mouthpiece of the device. An aerosol is a mixture of gas and solid or liquid particles. The most common nebulizers are jet nebulizers, sometimes referred to as atomizers. Other forms of nebulizers are soft mist inhalers, ultrasonic wave nebulizers, and vibrating mesh nebulizers. Nebulizers are disclosed in U.S. Pat. No. 10,799,902 to Maeda et al., U.S. Pat. No. 10,786,638 to Alizoti et al., and U.S. Pat. No. 10,716,907 to Eicher et al.

When active agents such as those described above are formulated for parenteral administration, e.g., formulated for injection via the intravenous, intramuscular, subcutaneous, intralesional, or intraperitoneal routes or other routes known in the art, many options are possible. The preparation of an aqueous composition that contains an effective amount of the active agent will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified. The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

Solutions of the active agents as free base or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and use, these preparations contain a preservative to prevent the growth of microorganisms. Suitable non-sensitizing and non-allergenic preservatives are well known in the art.

The carrier can also be a solvent and/or dispersion medium containing, for example, water, ethanol, a polyol (for example, glycerol, propylene glycol, and/or liquid polyethylene glycol, and/or the like), suitable mixtures thereof, and/or vegetable oils. The proper fluidity can be maintained for example, by the use of a coating, such as lecithin, by the maintenance of a suitable particle size in the case of a dispersion, and/or by the use of surfactants. The prevention of the action of microorganisms can be brought about by the inclusion of various antibacterial and/or antifungal agents, for example, parabens, chlorobutanol, phenol, sorbic acid, or thimerosal. In many cases it will be preferable to include isotonic agents, for example, sugars or sodium chloride. In many cases, it is preferable to prepare the solution in physiologically compatible buffers such as Hanks's solution, Ringer's solution, or physiological saline buffer. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by sterilization if desired or necessary. Sterilization is typically performed by filtration. However, alternative methods of sterilization are known in the art, such as by heating or autoclaving, if the ingredients in the solution are stable to such conditions and do not degrade or oxidize under such conditions. Generally, dispersions are prepared by incorporating the various sterilized active ingredients into a sterile vehicle which contains the basic dispersion medium and/or the other required ingredients. In the case of sterile powders for the preparation of sterile injectable solutions, the preferred methods of preparation are vacuum-drying and/or freeze-drying techniques that yield a powder of the active ingredients plus any additional desires ingredients from a previously sterile-filtered solution thereof. The preparation of more-concentrated or highly-concentrated solutions for direct injection is also contemplated, where the use of dimethyl sulfoxide (DMSO) as solvent is envisioned to result in extremely rapid penetration, delivering high concentrations of the active agents to a small area if desired.

For parenteral administration of an aqueous solution, for example, the solution should be suitably buffered if necessary and/or the liquid diluent first rendered isotonic with sufficient saline, glucose, or other tonicity agent. These particular aqueous solutions are especially suitable for intravenous, intramuscular, subcutaneous, or intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected into the proposed site of infusion (see, e.g., “Remington's Pharmaceutical Sciences” (15^{th} ed.), pp. 1035-1038, 1570-1580). Some variation in dosage will necessarily occur depending on the condition of the subject being treated. The person responsible for administration will, in any event, determine the appropriate dose for the individual subject. Compounds and compositions according to the invention can also be formulated for parenteral administration by bolus injection or continuous infusion and can be presented in unit dose form, for instance as ampules, vials, small volume infusions, or pre-filled syringes, or in multi-dose containers with an added preservative.

Another route of administration of active agents according to the present invention as described above is nasally, using dosage forms such as nasal solutions, nasal sprays, aerosols, or inhalants. Nasal solutions are usually aqueous solutions designed to be administered to the nasal passages in drops or sprays. Nasal solutions are typically prepared so that they are similar in many respects to nasal secretions, so that normal ciliary action is maintained. Thus, the aqueous nasal solutions usually are isotonic and/or slightly buffered in order to maintain a pH of from about 5.5 to about 6.5. In addition, antimicrobial preservatives, similar to those used in ophthalmic preparations, and/or appropriate drug stabilizers, if required, can be included in the formulation. Various commercial nasal preparations are known and can include, for example, antibiotics or antihistamines. Spray compositions can be formulated, for example, as aqueous solutions or suspensions or as aerosols delivered from pressurized packs, with the use of a suitable propellant, such as dichlorodifluoromethane, trichlorofluoromethane, dichlorotetrafluoroethane, 1,1,1,2,3,3,3-heptafluoropropane, 1,1,1,2-tetrafluoroethane, carbon dioxide, or other suitable gas.

Additional formulations that are suitable for other modes of administration include vaginal suppositories and/or pessaries. A rectal pessary or suppository can also be used. Suppositories are solid dosage forms of various weights or shapes, usually medicated, for insertion into the rectum, vagina, or urethra. After insertion, suppositories soften, melt, and/or dissolve into the cavity fluids. In general, for suppositories, conventional binders or carriers can include polyalkylene glycols, cocoa butter, or triglycerides.

Other dosage forms, including but not limited to liposomal formulations, ointments, creams, lotions, powders, or creams, can alternatively be used. Ointments and creams can, for example, be formulated with an aqueous or oily base with the addition of suitable gelling agents and/or solvents. Such bases, can thus, for example, include water and/or an oil such as liquid paraffin or a vegetable oil such as arachis (peanut) oil or castor oil or a solvent such as a polyethylene glycol. Thickening agents which can be used include soft paraffin, aluminum stearate, cetostearyl alcohol, polyethylene glycols, microcrystalline wax, and beeswax. Lotions can be formulated with an aqueous or oily base and will in general also contain one or emulsifying agents, stabilizing agents, dispersing agents, suspending agents, or thickening agents.

Powders for external application can be formed with the aid of any suitable powder base, for example, talc, lactose, or starch.

In some alternatives according to the present invention, one or more active agents can be administered as aerosols. Aerosol therapy allows an almost ideal benefit to risk ratio to be achieved because very small doses of inhaled medication provide optimal therapy with minimal adverse effects. However, the therapeutic efficiency of drugs administered by aerosolization depends not only on the pharmacological properties of the drugs themselves, but also on the characteristics of the delivery device. The characteristics of the delivery device influence the amount of drug deposited in the lungs and the pattern of drug distribution in the airways.

Aerosols are airborne suspensions of fine particles. The particles may be solids or liquids. Aerosol particles are heterodisperse (i.e. the particles are of a range of sizes) and aerosol particle size distribution is best described by a log normal distribution. Particles tend to settle (sediment), adhere to each other (coagulate), and adhere to structures such as tubing and mucosa (deposit). The particles delivered by aerosol can be conveniently characterized on the basis of their aerodynamic behavior. One parameter is the mass median aerodynamic diameter (MMAD). By definition, a particle distribution with an MMAD of 1 μm has the same average rate of settling as a droplet of unit density and 1 μm diameter.

The size of an aerosol particle, as well as variables affecting the respiratory system, influence the deposition of inhaled aerosols in the airways. On one hand, particles larger than 10 μm in diameter are unlikely to deposit in the lungs. However, particles smaller than 0.5 μm are likely to reach the alveoli or may be exhaled. Therefore, particles that have a diameter of between 1 μm and 5 μm are most efficiently deposited in the lower respiratory tract.

The percentage of the aerosol mass contained within respirable droplets (i.e., droplets with a diameter smaller than 5 μm), depends on the inhalation device being used. Slow, steady inhalation increases the number of particles that penetrate the peripheral parts of the lungs. As the inhaled volume is increased, the aerosol can penetrate more peripherally into the bronchial tree. A period of breath-holding, on completion of inhalation, enables those particles that have penetrated to the lung periphery to settle into the airways via gravity. Increased inspiratory flow rates, typically observed in patients with acute asthma, result in increased losses of inhaled drug. This occurs because aerosol particles impact in the upper airway and at the bifurcations of the first few bronchial divisions. Other factors associated with pulmonary airway disease may also alter aerosol deposition.

In aerosol administration, the nose efficiently traps particles before their deposition in the lung; therefore, mouth breathing of the aerosolized particles is preferred. The aerosolized particles are lost from many sites. Generally, the amount of the nebulized dose reaching the small airways is s 15%. In many cases, approximately 90% of the inhaled dose is swallowed and then absorbed from the gastrointestinal tract. The small fraction of the dose that reaches the airways is also absorbed into the blood stream. The swallowed fraction of the dose is, therefore, absorbed and metabolized in the same way as an oral formulation, while the fraction of the dose that reaches the airways is absorbed into the blood stream and metabolized in the same way as an intravenous dose.

When drugs are administered topically (via aerosol delivery to the lungs), the desired therapeutic effects depend on local tissue concentrations, which may not be directly related to plasma drug concentrations. This has several implications. First, for the selection of a drug to be inhaled, topical drugs must combine a high intrinsic activity within the target organ and rapid inactivation of the systemically absorbed drug. Secondly, fewer systemic adverse effects should be expected with drugs that have a low oral bioavailability (whether due to poor gastrointestinal absorption or high first-pass hepatic metabolism). Because most inhaled drugs are administered at a low dosage and have a low oral bioavailability, plasma concentrations of these drugs are much lower than after oral administration. Furthermore, factors influencing pulmonary absorption should be considered.

All jet nebulizers work via a similar operating principle, represented by the familiar perfume atomizer. A liquid is placed at the bottom of a closed container, and the aerosol is generated by a jet of air from either a compressor or a compressed gas cylinder passing through the device. Ultrasonic nebulizers produce an aerosol by vibrating liquid lying above a transducer at frequencies of about 1 mHz. This produces a cloud of particles that is carried out of the device to the patient by a stream of air. Aerosols varying in quantity, size and distribution of panicles can be produced by nebulizers, depending upon the design of the nebulizers and how they are operated. It should be noted that not all nebulizers have the required specifications (MMAD, flow, output) to provide optimum efficacy. A recent study compared the lung deposition from 4 nebulizers in healthy volunteers and showed that median lung aerosol deposition, expressed as percentages of the doses initially loaded into the nebulizers, ranged from 2 to 19%.

Metered dose inhalers (MDIs), because of their convenience and effectiveness, are probably the most widely used therapeutic aerosol used for inhaled drug delivery to outpatients. Most MDIs in current use contain suspensions of drug in propellant. There are 2 major components of an MDI: (i) the canister, a closed plastic or metal cylinder that contains propellant, active medication, and the metering chamber; and (ii) the actuator, a molded plastic container that holds the canister and directs the released aerosol towards the patient's airway.

Propellant mixtures are selected to achieve the vapor pressure and spray characteristics desired for optimal drug delivery. Chlorofluorocarbons were previously used, but non-chlorinated propellants are now employed because of environmental concerns. Finely divided particles of drug, usually less than 1 μm, are suspended in the pressurized (liquefied) propellant. To prevent the drug from coagulating, a surface-active agent such as sorbitan oleate, lecithin or oleic acid is typically added; other surface-active agents are known in the art. Metering chambers ordinarily contain 25 to 100 μL. The contents of the metering chamber are released when the canister is depressed into the actuator. Almost instantaneously, the propellants begin to evaporate, producing disintegration of the discharged liquid into particles that are propelled forward with great momentum. For optimal pulmonary drug deposition, the medication should be released at the beginning of a slow inspiration that lasts about 5 seconds and is followed by 10 seconds of breath-holding. Several inhalation aids have been designed to improve the effectiveness of an MDI. These are most useful in patients who have poor hand-to-breath coordination. A short tube (e.g. cones or spheres) may direct the aerosol straight into the mouth or collapsible bags may act as an aerosol reservoir holding particles in suspension for 3 to 5 seconds, during which time the patient can inhale the drug. However, when any of these devices is used, aerosol velocity upon entering the oropharynx is decreased and drug availability to the lungs and deposition in the oropharynx is decreased.

Dry powder inhalers have been devised to deliver agents to patients who have difficulty using an MDI (such as children and elderly patients). In general, the appropriate dosage is placed in a capsule along with a flow aid or filler such as large lactose or glucose panicles. Inside the device, the capsule is initially either pierced by needles (e.g., Spinhaler®) or sheared in half (e.g., Rotohaler®). During inhalation the capsule rotates or a propeller is turned, creating conditions that cause the contents of the capsule to enter the inspired air and be broken up to small particles suitable for delivery to the airways. The energy required to disperse the powder is derived from the patient's inspiratory effort. Recently, more convenient multidose dry powder inhalers have been introduced (e.g. Diskhaler®, Turbuhaler®). Potential problems associated with dry powder inhalers include esophageal irritation and, consequently, cough due to the direct effect of powder in airways. Furthermore, the walls of the capsule may be coated with drug as a result of either failure of the capsule to release the drug or failure of the aggregated powder to break up. This may cause virtually all of the drug to be deposited in the mouth. These powder devices do not contain chlorofluorocarbons and may provide an alternative to MDIs.

To ensure maximal effects from inhaled drugs, both the pharmacological characteristics of the drugs and the device used to aerosolize the drugs should be considered. A unit dose from a dry powder inhaler is twice that released from an MDI, but they have equivalent bronchodilatory effects. The characteristics of the devices vary. For a metered-dose inhaler, typically 12-40% of the dose is deposited in the lung, but up to 80% in the oropharynx. When an MDI is used with a spacer, typically about 20% of the dose is deposited in the lung, but only up to 5% in the oropharynx; thus, the use of a spacer can reduce the proportion of the drug that is deposited in the oropharynx. For a dry powder inhaler, typically 11-16% of the dose is deposited in the lung and 31-72% in the oropharynx. For a nebulizer, typically 7-32% of the dose is deposited in the lung and 1-9% is deposited in the oropharynx. One of ordinary skill in the art can ensure that the proper inhalation therapy device is used and can prepare suitable instructions. Considerations for the use of inhalation therapy are described in A.-M. Tabaret & B. Schmit, “Pharmacokinetic Optimisation of Asthma Treatment,” Clin. Pharmacokinet. 26: 396-418 (1994).

In one particular alternative for a formulation of the Form A polymorph of valomaciclovir stearate to be administered orally in caplet form, the formulation comprises, per caplet: 1000 mg of the Form A polymorph of valomaciclovir stearate; 180.53 mg of croscarmellose sodium; 42.31 mg of Povidone K-30; 13.08 mg of polysorbate 80; 53.21 mg of talc; and 9.60 mg of magnesium stearate. The formulation is prepared by granulation and blending.

Another aspect of the present invention is a method for treating long COVID. Several studies have indicated that about 20% of patients with acute COVID develop long COVID. Although there is a hypothesis that long COVID could be caused by persistent reservoirs of SARS-CoV-2 virus, several recent publications have indicated that persistent SARS-CoV-2 virus is not found in such patients but evidence of persistent Epstein-Barr virus infection does exist in such patients.

Although Applicant is not bound by this theory, Applicant believes that SARS-CoV-2 virus acute infection can impair natural immunity of the host including cell-mediated immunity (CMI). Because CMI holds latent herpesvirus in latency, impairment of CMI by SARS-CoV-2 virus infection and the stress of acute COVID can lead to reactivation of herpesviruses such as Epstein-Barr virus, HHV6a, and HHV6b and cause lytic replication of these viruses. Lytic replication of Epstein-Barr virus in acute infectious mononucleosis can lead to symptoms of fatigue, brain fog, sleep disturbances, and myalgias. These symptoms associated with acute infectious mononucleosis are very similar to symptoms occurring in long COVID. Accordingly, Applicant believes that the main symptoms of long COVID are caused by reactivated Epstein-Barr virus. There may also be a subgroup of long COVID patients whose symptoms are caused both by persistent reservoirs of SARS-CoV-2 virus infection and reactivation of a herpesvirus such as Epstein-Barr virus, HHV6a, or HHV6b, or by reactivation of multiple herpesviruses.

Accordingly, a method for triple therapy of long COVID employs induction of herpesviruses such as Epstein-Barr virus, antiviral agents, and anti-B-cell therapy such as an anti-CD20 agent, an anti-integrin α4β1 agent, or an anti-CD37 agent as described above. The triple therapy proceeds in three stages.

The induction agents can include, but are not limited to, ribavirin, typically administered at 400 mg twice daily, arginine, typically administered at about 2 g to about 3 g twice daily, and histone deacetylase (HDAC) inhibitors, including, but not limited to, valproic acid, sodium butyrate, romidepsin, abexinostat, belinostat, entinostat, givinostat, martinostat, mocetinostat, panobinostat, pracinostat, resminostat, sodium phenylbutyrate, tucidinostat, and vorinostat.

The anti-B-cell agents are as described above and include anti-CD20 agents, anti-integrin α4β1 agents, or anti-CD37 agents.

In this triple therapy method, antiviral therapy is initiated at a high dose by administration of: intravenous or intramuscular H2G (omaciclovir) at a dosage of about 5 mg/kg to about 25 mg/kg twice daily for 1 day to 6 weeks; or administration of a capsule or tablet polymorph of Form A of valomaciclovir stearate at about 1.5 g to 2 g orally twice daily for 1 week to 6 weeks; in addition, tenofovir intravenously can be administered at 5 mg to 150 mg twice daily for 1 day to 6 weeks, or, as an alternative, tenofovir disoproxil fumarate can be administered at about 5 mg to about 150 mg orally twice daily (or in a combination pill with polymorph Form A of valomaciclovir stearate), and pharmaceutical grade cod liver oil can be administered at about 80 mg to about 500 mg twice daily for 2 to 6 weeks. The antiviral therapy is continued at a low dose by administration of polymorph Form A of valomaciclovir stearate at about 500 mg to about 850 mg, tenofovir disoproxil fumarate at about 75 mg, and pharmaceutical grade cod liver oil at about 80 mg to about 500 mg twice daily chronically. Another alternative for chronic low dose therapy is administration of polymorph Form A of valomaciclovir stearate at about 500 mg to about 850 mg, tenofovir disoproxil fumarate at about 75 mg, and sildenafil at about 5 mg to about 10 mg twice daily. All of the techniques and combinations of valomaciclovir and H2G recited herein for other indications can be applied to treating long COVID.

For use in these methods, tablets or capsules can be prepared. One alternative of a tablet or capsule for use in both the high-dose antiviral therapy and the low-dose antiviral therapy comprises polymorph Form A of valomaciclovir stearate at about 500 mg to about 850 mg, tenofovir disoproxil fumarate at about 75 mg, and pharmaceutical grade cod liver oil at about 80 mg to about 200 mg. Another alternative of a tablet or capsule for use in the low-dose antiviral therapy comprises polymorph Form A of valomaciclovir stearate at about 500 mg to about 850 mg, tenofovir disoproxil fumarate at about 75 mg, and sildenafil at about 5 mg to about 20 mg.

Another aspect of therapy according to the present invention is a method for treating long COVID cases in which both persistent SARS-CoV-2 virus and reactivated Epstein-Barr virus or reactivated HHV6 virus are of concern. In general, this method comprises administering a combination of about 75 mg of nirmatrelvir, about 50 mg of ritonavir, and about 750 mg of polymorph Form A of valomaciclovir stearate twice daily for chronic treatment.

Yet another aspect of the present invention is a combination pill, the combination pill comprising about 10 mg to about 75 mg of nirmatrelvir per unit dose, about 10 mg to about 50 mg of ritonavir per unit dose, and about 750 mg of polymorph Form A of valomaciclovir stearate per unit dose. This combination pill is particularly suitable for use in the method described above for treating long COVID cases in which both persistent SARS-CoV-2 virus and reactivated Epstein-Barr virus or reactivated HHV6 virus are of concern. This combination pill can further comprise suitable carriers or excipients as described above.

One aspect of the present invention is a method for treating relapsing-remitting multiple sclerosis or HLA marker DR2 (DRB1*1501) progressive multiple sclerosis, the method comprising the steps of:
(1) administering H2G (omaciclovir) intravenously or intramuscularly at a dosage of about 5 mg/kg to about 25 mg/kg twice daily for 1 day to 6 weeks;
(2) subsequently administering high-dose valomaciclovir stearate for 1 week to 6 weeks at a dosage of about 1.5 g to about 2 g orally twice daily; and
(3) then subsequently administering chronic low-dose valomaciclovir stearate at a dosage of about 1 g to about 2 g orally with chronic dosing.

Preferably, in this methods and in all other methods recited in this application, the valomaciclovir stearate is polymorph Form A of valomaciclovir stearate. However, in some alternatives, another polymorphic form can be used.

In one alternative for this method, and for other methods recited in this application when H2G is administered, intramuscular injection for H2G may be administered by a pre-loaded auto-injector.

Yet another aspect of the present invention is a method for treating relapsing-remitting multiple sclerosis or HLA marker DR2 (DRB1*1501) progressive multiple sclerosis, the method comprising the steps of:
(1) subsequently administering high-dose valomaciclovir stearate for 1 week to 6 weeks at a dosage of about 1.5 g to about 2 g orally twice daily; and
(2) then subsequently administering chronic low-dose valomaciclovir stearate at a dosage of about 1 g to about 2 g orally with chronic dosing.

Another aspect of the present invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks with tenofovir at about 5 mg to about 150 mg in the H2G intravenous or intramuscular solution or administered as a separate intravenous bolus or intramuscular injection for 1 day to 6 weeks or with tenofovir disoproxil fumarate administered orally at about 10 mg to about 150 mg twice daily;
(2) subsequently administering high-dose valomaciclovir stearate at about 1.5 g to about 2 g twice daily orally combined with tenofovir disoproxil fumarate, wherein the tenofovir disoproxil fumarate is administered separately orally or in the form of a combination tablet or capsule containing both valomaciclovir stearate and tenofovir disoproxil fumarate; and
(3) subsequently administering a low dose valomaciclovir stearate therapy of about 1 g to 2 g daily orally with chronic dosing.

In one alternative of this method, when valomaciclovir stearate and tenofovir or tenofovir disoproxil fumarate are administered, the therapy can include an additional agent selected from the group consisting of an agent to improve penetration of the blood-brain barrier, an agent to improve gastrointestinal absorption, or an agent to prevent reactivation of herpesvirus.

Yet another aspect of the present invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks plus low-dose interferon type 1, either Avonex (interferon beta-1a) once weekly intramuscularly at 7.5 μg once in week 1 and 15 μg once in week 2, and 30 μg once in weeks 3 and 4 or Betaseron (interferon beta-1 b) every other day subcutaneously with 0.0625 mg in week 1, 0.125 mg in week two; 0.1875 mg in week 3; and 0.25 mg in week 4;
(2) subsequently administering high-dose valomaciclovir stearate at about 1.5 g to about 2 g orally twice daily combined with low-dose interferon type 1 therapy as described in step (1); and
(3) then followed by low-dose valomaciclovir stearate orally at 1 g to 2 g once daily with chronic dosing.

Still another aspect of the present invention is a method for treating relapsing-remitting multiple sclerosis or HLA marker DR2 (DRB1*1501) progressive multiple sclerosis, the method comprising the steps of:
(1) administering high-dose valomaciclovir stearate at about 1.5 g to about 2 g orally twice daily combined with low-dose interferon type 1 therapy, either Avonex (interferon beta-1a) once weekly intramuscularly at 7.5 μg once in week 1 and 15 μg once in week 2, and 30 μg once in weeks 3 and 4 or Betaseron (interferon beta-1 b) every other day subcutaneously with 0.0625 mg in week 1, 0.125 mg in week two; 0.1875 mg in week 3; and 0.25 mg in week 4; and
(2) subsequently followed by low-dose valomaciclovir stearate orally at 1 g to 2 g once daily with chronic dosing.

Yet another aspect of the invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks plus low-dose interferon type 1 twice daily as described above for interferon 1a and interferon-1b plus tenofovir intravenously or intramuscularly at 5 mg to 150 mg twice daily for 1 day to 6 weeks or tenofovir disoproxil fumarate at about 150 mg twice daily for 1 day to 6 weeks; and
(2) subsequently administering high-dose valomaciclovir stearate at about 1.5 g to 2 g twice daily combined with low-dose interferon type 1 (as described above) and low-dose tenofovir disoproxil fumarate orally once daily.

Still another aspect of the invention is a method for treating relapsing-remitting multiple sclerosis or HLA marker DR2 (DRB1*1501) progressive multiple sclerosis, the method comprising the steps of:
(1) administering H2G intravenously or intramuscularly at about 5 mg/kg to about 25 mg/kg twice daily for 1 day to 6 weeks or administering valomaciclovir stearate orally in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; and (iii) mycophenolate administered orally or intravenously at about 500 mg to about 1.5 mg twice daily for 1 day to 6 weeks; and
(2) subsequently administering chronic low-dose valomaciclovir stearate at a dosage of about 1 g to about 2 g once daily.

In the above aspects of the present invention, the method can further comprises administration of a therapeutically effective quantity of an additional anti-integrin α4β1 agent such as natalizumab. In another alternative of the present invention, the method further comprises administration of a therapeutically effective quantity of an additional anti-CD20 agent, such as an anti-CD20 agent selected from the group consisting of rituximab, ocrelizumab, obinutuzumab, ofatumumab, ibritumomab tiuxetan, tositumomab, and ublituximab. In yet another alternative of the present invention, the method further comprises administration of a therapeutically effective quantity of an additional anti-CD37 agent. The anti-CD37 agent can be selected from the group consisting of lilotomab satetrextan, Humalutin, naratuximab emtansine, and otlertuzumab.

Still another aspect of the invention is a method for preventing reactivation of Epstein-Barr virus comprising the step of administering a therapeutically effective quantity of valomaciclovir stearate in pill form together with oral lysine in a combination pill, wherein the quantity of oral lysine administered is about 200 mg to about 500 mg twice daily. The combination pill can further comprise a therapeutically effective quantity of H2G. The method can further comprise administering minoxidil at about 5 mg to about 40 mg orally twice daily, administering sildenafil at about 5 mg to about 20 mg orally twice daily, or administering vardenafil at about 5 mg to about 20 mg orally twice daily. As stated above, preferably, the valomaciclovir stearate is polymorph Form A of valomaciclovir stearate. The valomaciclovir stearate can be administered orally in pill form together with an agent selected from the group consisting of minoxidil, sildenafil, and vardenafil.

In other alternatives according to the present invention, administration of intravenous H2G is followed by intranasal administration of a combination H2G/tenofovir spray twice daily.

Yet another aspect of the present invention is a method for treating a disease or condition selected from the group consisting of acute infectious mononucleosis, chronic mononucleosis, long COVID, chronic fatigue syndrome, fibromyalgia, Crohn's disease, ulcerative colitis, rheumatoid arthritis, SLE, Graves' disease, Alzheimer's disease, mesial temporal lobe seizures, Epstein-Barr-virus-linked autism, and an Epstein-Barr-virus-linked cancer, the method comprising the steps of:
(1) administering H2G intravenously or intramuscularly at about 5 mg/kg to about 25 mg/kg twice daily or administering valomaciclovir stearate polymorph Form A orally at a high dose of about 1.5 g to 2 g orally twice daily in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; and (iii) mycophenolate administered orally or intravenously at about 500 mg to about 1.5 mg twice daily for 1 day to 6 weeks;
(2) subsequently administering low-dose valomaciclovir stearate polymorph Form A at a dosage of about 1 g to 2 g once daily with chronic dosing; and
(3) then subsequently administering a therapeutically effective quantity of an additional agent selected from the group consisting of an anti-CD20 agent, an anti-integrin α4β1 agent, and an anti-CD37 agent.

In this method, the disease or condition can be an Epstein-Barr-virus-linked cancer, and the Epstein-Barr-virus-linked cancer can be selected from the group consisting of nasopharyngeal carcinoma, glioblastoma multiforme, and Kaposi's sarcoma.

Still another aspect of the present invention is a method for treatment of glioblastoma comprising the steps of:
(1) administering an induction agent selected from the group consisting of:
   (a) ribavirin administered at about 100 mg to about 400 mg twice daily for 1 day to 4 weeks;
   (b) romidepsin administered at about 14 mg/m² on days 1 and 8; and
   (c) sodium phenylbutyrate administered at a loading dose of about 250 mg intravenously over 90 minutes, followed by a dose of about 250 mg/kg per day for 7 days; followed by
(2) administering H2G intramuscularly or intravenously at a dose of about 5 mg/kg to about 25 mg/kg twice daily for 1 day to 6 weeks; followed by
(3) administering artesunate at a dose of about 1 mg/kg to about 2.4 mg/kg for 1 day to 6 weeks; followed by
(4) administering high dose polymorph Form A of valomaciclovir stearate at about 1.5 g to about 2 g twice daily for 1 week to 6 weeks, tenofovir disoproxil fumarate at about 75 mg twice daily for 1 day to 6 weeks, with the dose depending on the patient's renal function, and sildenafil at about 5 mg twice daily for 1 day to 6 weeks; and followed by
(5) administering about 850 mg of polymorph Form A of valomaciclovir stearate twice daily, tenofovir disoproxil fumarate at about 75 mg twice daily, with the dose and dosing duration depending on the patient's renal function with chronic dosing, and sildenafil at about 5 mg to about 20 mg with twice daily chronic dosing.

Yet another aspect of the invention is a formulation of polymorph Form A of valomaciclovir stearate for oral administration, the formulation comprising, per unit dose:
(1) 1000 mg of the Form A polymorph of valomaciclovir stearate;
(2) 180.53 mg of croscarmellose sodium;
(3) 42.31 mg of Povidone K-30;
(4) 13.08 mg of polysorbate 80;
(5) 53.21 mg of talc; and
(6) 9.60 mg of magnesium stearate;
wherein the formulation is in the form of caplets.

Still another aspect of the present invention is a method for triple therapy of long COVID comprising the steps of:
(1) administering a therapeutically effective quantity of an induction agent for a human herpesvirus;
(2) administering a therapeutically effective quantity of at least one antiviral agent selected from the group consisting of H2G and polymorph Form A of valomaciclovir stearate, wherein the antiviral agent or agents are administered in two stages: a first high-dose stage and a second low-dose chronic stage; and
(3) administering a therapeutically effective quantity of an anti-B-cell agent selected from the group consisting of anti-CD20 agent, an anti-integrin α4β1 agent, or an anti-CD37 agent.

In this method, the induction agent can be selected from the group consisting of ribavirin, arginine, and an HDAC inhibitor; the HDAC inhibitor can be selected from the group consisting of valproic acid, sodium butyrate, romidepsin, abexinostat, belinostat, entinostat, givinostat, martinostat, mocetinostat, panobinostat, pracinostat, resminostat, sodium phenylbutyrate, tucidinostat, and vorinostat.

In this method, the antiviral therapy can be initiated at a high dose by administration of intravenous or intramuscular H2G (omaciclovir) at a dosage of about 5 mg/kg to about 25 mg/kg per day twice daily for 1 to 6 weeks. Alternatively, the antiviral therapy can be initiated at a high dose by administration of polymorph Form A of valomaciclovir stearate at about 1.5 g to 2 g orally twice daily, tenofovir disoproxil fumarate, and pharmaceutical grade cod liver oil at about 400 mg twice daily for 2 to 6 weeks. In this method, the antiviral therapy can then be continued at a low dose by administration of polymorph Form A of valomaciclovir stearate at about 500 mg to about 850 mg, tenofovir disoproxil fumarate at about 75 mg, and pharmaceutical grade cod liver oil at about 80 mg to about 500 mg twice daily with chronic dosing. Alternatively, the antiviral therapy can be continued at a low dose by administration of polymorph Form A of valomaciclovir stearate at about 500 mg to about 850 mg, tenofovir disoproxil fumarate at about 75 mg, and sildenafil at about 5 mg to about 20 mg twice daily with chronic dosing.

Still another aspect of the invention is a capsule or tablet formulation comprising:
(1) polymorph Form A of valomaciclovir stearate at about 500 mg to about 850 mg per capsule or tablet;
(2) tenofovir disoproxil fumarate at about 75 mg per capsule or table (with daily dosing of tenofovir disoproxil fumarate dependent on the patient's renal status); and
(3) pharmaceutical grade cod liver oil at about 80 mg to about 500 mg per capsule or tablet.

Yet another aspect of the invention is a capsule or tablet formulation comprising:
(1) polymorph Form A of valomaciclovir stearate at about 500 mg to about 850 mg per capsule or tablet;
(2) tenofovir disoproxil fumarate at about 75 mg per capsule or tablet (with daily dosing of tenofovir disoproxil fumarate dependent on the patient's renal status); and
(3) sildenafil at about 5 mg to about 20 mg per capsule or tablet.

Still another aspect of the invention is a method for treating long COVID cases in which both persistent SARS-CoV-2 virus and reactivated Epstein-Barr virus or reactivated HHV6 virus is of concern, the method comprising the steps of:
(1) chronically administering a therapeutically effective quantity of nirmatrelvir;
(2) chronically administering a therapeutically effective quantity of ritonavir; and
(3) chronically administering a therapeutically effective quantity of polymorph Form A of valomaciclovir stearate.

In this method, in more detail, the method can comprise the steps of:
(1) chronic administration of about 75 mg of nirmatrelvir twice daily;
(2) chronic administration of about 50 mg of ritonavir twice daily; and
(3) chronic administration of about 750 mg of polymorph Form A of valomaciclovir stearate twice daily.

Yet another aspect of the present invention is a combination pill comprising:
(1) about 75 mg of nirmatrelvir per unit dose;
(2) about 50 mg of ritonavir per unit dose; and
(3) about 750 mg of polymorph Form A of valomaciclovir stearate per unit dose.

It has recently been shown that remdesivir can reactivate latent Epstein-Barr virus (EBV). EBV is oncogenic and has also been linked to multiple sclerosis and long COVID. Recent publications have also postulated that reactivated EBV has been linked to long COVID. It has also been postulated that long COVID is caused by persistent SARS-CoV2.

Therefore, still another aspect of the present invention is a method of treating long COVID comprising the step of administering a therapeutically effective quantity of a combination of therapeutic agents selected from the group consisting of:
(1) valomaciclovir stearate and remdesivir, wherein the remdesivir is administered orally; and
(2) H2G and remdesivir, wherein the remdesivir is administered intravenously.

Typically, in this method, the valomaciclovir stearate is the polymorphic Form A of valomaciclovir stearate.

The indications for these combinations would be to prevent the development of long COVID in acute COVID cases and to treat long COVID. By combining valomaciclovir stearate and oral remdesivir, the combination therapy could suppress SARS-CoV2 without increasing EBV lytic activity since valomaciclovir stearate suppresses replication of EBV and yet valomaciclovir stearate does not impact CMV. This suggests effectiveness for treating or preventing long COVID.

In one alternative of this method, when remdesivir and valomaciclovir stearate are administered, the active agents are administered in a dosage form that is pill, tablet, or capsule comprising about 300 mg valomaciclovir stearate plus about 700 mg remdesivir in each 1-gram tablet. When this dosage form is administered, typically, the initial dose for long COVID would be two 1-gram tablets twice daily for 4 days to 6 weeks followed by chronic dosing of one tablet once daily or twice daily. In another alternative for use of this dosage form, the dosage form could be administered for 4 days to 6 weeks followed by once-daily dosing of either valomaciclovir stearate or a combination dosage form including valomaciclovir stearate, wherein the combination dosage form including valomaciclovir stearate is selected from: (i) a combination dosage form comprising a therapeutically effective quantity of valomaciclovir stearate and a therapeutically effective quantity of sildenafil; (ii) a combination dosage form comprising a therapeutically effective quantity of valomaciclovir stearate and a therapeutically effective quantity of tenofovir alafenamide fumarate; and (iii) a combination dosage form comprising a therapeutically effective quantity of valomaciclovir stearate and a therapeutically effective quantity of lysine. When the combination dosage form includes sildenafil, the sildenafil is included to improve blood-brain barrier (BBB) penetration. The combination dosage form could also be formulated as a liquid or nasal spray. The combination may also generate synergies and improve the efficacy of oral remdesivir in treating SARS-CoV2; it may also increase the anti-EBV effect of valomaciclovir stearate against EBV.

In the context of this combination therapy, yet another aspect of the present invention is a combination dosage form including valomaciclovir stearate, wherein the combination dosage form including valomaciclovir stearate is selected from: (i) a combination dosage form comprising a therapeutically effective quantity of valomaciclovir stearate and a therapeutically effective quantity of sildenafil; (ii) a combination dosage form comprising a therapeutically effective quantity of valomaciclovir stearate and a therapeutically effective quantity of tenofovir alafenamide fumarate; and (iii) a combination dosage form comprising a therapeutically effective quantity of valomaciclovir stearate and a therapeutically effective quantity of lysine. This combination dosage form can be formulated as a solid form, such as a pill, a capsule, or a tablet, or as a liquid or nasal spray.

Still another aspect of the present invention is a method of treating a disease or condition selected from the group consisting of long COVID, Alzheimer's disease, multiple sclerosis, chronic fatigue syndrome, fibromyalgia, acute infectious mononucleosis, and chronic mononucleosis comprising the step of administering a therapeutically effective quantity of valomaciclovir stearate and a therapeutically effective quantity of taurine. The therapeutically effective quantity of valomaciclovir stearate and the therapeutically effective quantity of taurine could be administered in a capsule, pill, tablet, liquid, nasal spray, or patch. Typically, in this method, the valomaciclovir stearate is the polymorphic Form A of valomaciclovir stearate. Taurine deficiency has been found in a recent study of long COVID patients. It has been proposed that taurine may improve brain fog or focus as well as improving short-term memory.

In the context of this combination therapy, yet another aspect of the present invention is a combination dosage form comprising a therapeutically effective quantity of valomaciclovir stearate and a therapeutically effective quantity of taurine. This combination dosage form can be formulated as a solid form, such as a pill, a capsule, or a tablet, or as a liquid or nasal spray, or as a patch.

### Advantages of the Invention

The present invention provides a new method for treating diseases and conditions associated with infection by and continued persistence of herpesviruses, particularly Epstein-Barr virus. These diseases and conditions include, but are not necessarily limited to, multiple sclerosis, chronic mononucleosis, long COVID, chronic fatigue syndrome, fibromyalgia, Crohn's disease, ulcerative colitis, rheumatoid arthritis, systemic lupus erythematosus, Graves' disease, Alzheimer's disease, mesial temporal lobe seizures, Epstein-Barr-virus-linked autism, and Epstein-Barr-virus-linked cancers including, but not limited to, nasopharyngeal carcinoma, glioblastoma multiforme, and Kaposi's sarcoma. The methods of the present invention are well-tolerated and can be used together with other methods known in the art for treating these diseases and conditions.

As used herein in the specification and claims, the transitional phrase “comprising” and equivalent language also encompasses the transitional phrases “consisting essentially of” and “consisting of” with respect to the scope of any claims presented herein, unless the narrower transitional phrases are explicitly excluded.

Methods according to the present invention possess industrial applicability for the preparation of a medicament for the treatment of these diseases and conditions. Methods according to the present invention also possess industrial applicability for use in treating these diseases and conditions. Recitation of a specific method also includes, where appropriate, recitation of a specific pharmaceutical composition or formulation for use in that method.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug. Methods as described herein also encompass use of the compounds, combinations of compounds, or compositions described herein for the treatment of the diseases or conditions described herein, as well as methods for the preparation of a medicament for the treatment of the diseases or conditions described herein.

The inventions illustratively described herein can suitably be practiced in the absence of any element or elements, limitation or limitations, not specifically disclosed herein. Thus, for example, the terms “comprising,” “including,” “containing,” and equivalent or similar terminology shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A method for treating relapsing-remitting multiple sclerosis or HLA marker DR2 (DRB1*1501) progressive multiple sclerosis, the method comprising the steps of:
(a) administering H2G (omaciclovir) intravenously or intramuscularly at a dosage of about 5 mg/kg to about 25 mg/kg twice daily for 1 day to 6 weeks;
(b) subsequently administering high-dose valomaciclovir stearate for 1 week to 6 weeks at a dosage of about 1.5 g to about 2 g orally twice daily; and
(c) then subsequently administering chronic low-dose valaciclovir stearate at a dosage of about 1 g to about 2 g orally with chronic dosing.

2. The method of claim 1 wherein the valomaciclovir stearate is polymorph Form A of valomaciclovir stearate.

3. The method of claim 1 wherein the H2G is administered intramuscularly by a pre-loaded auto-injector.

4. A method for treating relapsing-remitting multiple sclerosis or HLA marker DR2 (DRB1*1501) progressive multiple sclerosis, the method comprising the steps of:
(a) subsequently administering high-dose valomaciclovir stearate for 1 week to 6 weeks at a dosage of about 1.5 g to about 2 g orally twice daily; and
(b) then subsequently administering chronic low-dose valomaciclovir stearate at a dosage of about 1 g to about 2 g orally with chronic dosing.

5. The method of claim 4 wherein the valomaciclovir stearate is polymorph Form A of valomaciclovir stearate.

6. A method for treating relapsing-remitting multiple sclerosis or HLA marker DR2 (DRB1*1501) progressive multiple sclerosis, the method comprising the steps of:
(a) administering H2G intravenously or intramuscularly at about 5 mg/kg to about 25 mg/kg twice daily for 1 day to 6 weeks with tenofovir at about 5 mg to about 150 mg in the H2G intravenous solution or administered as a separate intravenous bolus or intramuscular injection for 1 day to 6 weeks or with tenofovir disoproxil fumarate administered orally at about 10 mg to about 150 mg twice daily;
(b) subsequently administering high-dose valomaciclovir stearate at about 1.5 g to about 2 g twice daily orally combined with tenofovir disoproxil fumarate, wherein the tenofovir disoproxil fumarate is administered separately orally or in the form of a combination tablet or capsule containing both valomaciclovir stearate and tenofovir disoproxil fumarate; and
(c) subsequently administering a low dose valomaciclovir stearate therapy of about 1 g to 2 g daily orally with chronic dosing.

7. The method of claim 6 wherein the valomaciclovir stearate is polymorph Form A of valomaciclovir stearate.

8. The method of claim 6 wherein, in step (a), the tenofovir is administered in the H2G intravenous solution.

9. The method of claim 6 wherein, in step (a), the tenofovir is administered as a separate intravenous bolus or intramuscular injection.

10. The method of claim 6 wherein, in step (a), the tenofovir disoproxil fumarate is administered orally.

11. The method of claim 6 wherein, in step (b), the tenofovir disoproxil fumarate is administered separately orally.

12. The method of claim 6 wherein, in step (b), the tenofovir disoproxil fumarate is administered in the form of a combination tablet or capsule.

13. The method of claim 6 wherein the method further comprises administration of an additional agent selected from the group consisting of an agent to improve penetration of the blood-brain barrier, an agent to improve gastrointestinal absorption, and an agent to prevent reactivation of herpesvirus.

14. The method of claim 13 wherein the additional agent is an agent to improve penetration of the blood-brain barrier, and wherein the agent to improve penetration of the blood-brain barrier is selected from the group consisting of minoxidil, sildenafil, and vardenafil.

15. The method of claim 13 wherein the additional agent is an agent to improve gastrointestinal absorption, and wherein the agent to improve gastrointestinal absorption is pharmaceutical grade cod liver oil.

16. The method of claim 13 wherein the additional agent is an agent to prevent reactivation of herpesvirus, and wherein the agent to prevent reactivation of herpesvirus is lysine.

17. A method for treating relapsing-remitting multiple sclerosis or HLA marker DR2 (DRB1*1501) progressive multiple sclerosis, the method comprising the steps of:
(a) administering H2G intravenously or intramuscularly at about 5 mg/kg to about 25 mg/kg twice daily for 1 day to 6 weeks plus low-dose interferon type 1, either Avonex (interferon beta-1a) once weekly intramuscularly at 7.5 μg once in week 1 and 15 μg once in week 2, and 30 μg once in weeks 3 and 4 or Betaseron (interferon beta-1 b) every other day subcutaneously with 0.0625 mg in week 1, 0.125 mg in week two; 0.1875 mg in week 3; and 0.25 mg in week 4;
(b) subsequently administering high-dose valomaciclovir stearate at about 1.5 g to about 2 g orally twice daily combined with low-dose interferon type 1 therapy as described in step (a); and
(c) then followed by low-dose valomaciclovir stearate orally at 1 g to 2 g once daily with chronic dosing.

18. The method of claim 17 wherein the valomaciclovir stearate is polymorph Form A of valomaciclovir stearate.

19. The method of claim 17 wherein the interferon-1 is interferon-1a.

20. The method of claim 17 wherein the interferon-1 is interferon-1 b.

21. A method for treating relapsing-remitting multiple sclerosis or HLA marker DR2 (DRB1*1501) progressive multiple sclerosis, the method comprising the steps of:
(a) administering H2G intravenously or intramuscularly at about 5 mg/kg to about 25 mg/kg twice daily for 1 day to 6 weeks plus low-dose interferon type 1, either Avonex (interferon beta-1a) once weekly intramuscularly at 7.5 μg once in week 1 and 15 μg once in week 2, and 30 μg once in weeks 3 and 4 or Betaseron (interferon beta-1 b) every other day subcutaneously with 0.0625 mg in week 1, 0.125 mg in week two; 0.1875 mg in week 3; and 0.25 mg in week 4; and
(b) subsequently followed by low-dose valomaciclovir stearate orally at 1 g to 2 g once daily with chronic dosing.

22. The method of claim 21 wherein the valomaciclovir stearate is polymorph Form A of valomaciclovir stearate.

23. The method of claim 21 wherein the interferon-1 is interferon-1a.

24. The method of claim 21 wherein the interferon-1 is interferon-1 b.

25. A method for treating relapsing-remitting multiple sclerosis or HLA marker DR2 (DRB1*1501) progressive multiple sclerosis, the method comprising the steps of:
(a) administering H2G intravenously or intramuscularly at about 5 mg/kg to about 25 mg/kg twice daily for 1 day to 6 weeks, plus low-dose interferon type 1, either Avonex (interferon beta-1a) once weekly intramuscularly at 7.5 μg once in week 1 and 15 μg once in week 2, and 30 μg once in weeks 3 and 4 or Betaseron (interferon beta-1 b) every other day subcutaneously with 0.0625 mg in week 1, 0.125 mg in week two; 0.1875 mg in week 3; and 0.25 mg in week 4, plus tenofovir intravenously or intramuscularly at 5 mg to 150 mg twice daily for 1 day to 6 weeks or tenofovir disoproxil fumarate at about 150 mg twice daily for 1 day to 6 weeks; and
(b) subsequently administering high-dose valomaciclovir stearate at about 1.5 g to 2 g twice daily combined with low-dose interferon type 1 (as described above) and low-dose tenofovir disoproxil fumarate orally once daily.

26. The method of claim 25 wherein the valomaciclovir stearate is polymorph Form A of valomaciclovir stearate.

27. The method of claim 25 wherein the interferon-1 is interferon-1a.

28. The method of claim 25 wherein the interferon-1 is interferon-1 b.

29. The method of claim 25 wherein the method comprises administration of tenofovir.

30. The method of claim wherein the method comprises administration of tenofovir disoproxil fumarate.

31. A method for treating relapsing-remitting multiple sclerosis or HLA marker DR2 (DRB1*1501) progressive multiple sclerosis, the method comprising the steps of:
(a) administering H2G intravenously or intramuscularly at about 5 mg/kg to about 25 mg/kg twice daily for 1 day to 6 weeks or administering valomaciclovir stearate orally in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; and (iii) mycophenolate administered orally or intravenously at about 500 mg to about 1.5 mg twice daily for 1 day to 6 weeks; and
(b) subsequently administering chronic low-dose valomaciclovir stearate at a dosage of about 1 g to about 2 g once daily.

32. The method of claim 31 wherein the valomaciclovir stearate is polymorph Form A of valomaciclovir stearate.

33. The method of claim 31 wherein the additional agent is artesunate.

34. The method of claim 31 wherein the additional agent is ribavirin.

35. The method of claim 31 wherein the additional agent is mycophenolate.

36. The method of any one of claims 1, 4, 6, 17, 21, 25, and 31 wherein the method further comprises administration of a therapeutically effective quantity of an anti-integrin α4β1 agent, an anti-CD20 agent, or an anti-CD37 agent.

37. The method of claim 36 wherein the method comprises administration of a therapeutically effective quantity of an anti-integrin α4β1 agent, and wherein the anti-integrin α4β1 agent is natalizumab.

38. The method of claim 36 wherein the method comprises administration of a therapeutically effective quantity of an anti-CD20 agent, and wherein the anti-CD20 agent is selected from the group consisting of rituximab, ocrelizumab, obinutuzumab, ofatumumab, ibritumomab tiuxetan, tositumomab, and ublituximab.

39. The method of claim 36 wherein the method comprises administration of a therapeutically effective quantity of an anti-CD37 agent, and wherein the anti-CD37 agent is selected from the group consisting of lilotomab satetrextan, Humalutin, naratuximab emtansine, and otlertuzumab.

40. A method for preventing reactivation of Epstein-Barr virus comprising the step of administering a therapeutically effective quantity of valomaciclovir stearate in pill form together with oral lysine in a combination pill, wherein the quantity of oral lysine administered is about 200 mg to about 500 mg twice daily.

41. The method of claim 40 wherein the valomaciclovir stearate is polymorph Form A of valomaciclovir stearate.

42. The method of claim 40 wherein the combination pill further comprises a therapeutically effective quantity of H2G.

43. The method of claim 40 wherein the method further comprises administering minoxidil at about 5 mg to about 40 mg orally twice daily.

44. The method of claim 40 wherein the method further comprises administering sildenafil at about 5 mg to about 20 mg orally twice daily.

45. The method of claim 40 wherein the method further comprises administering vardenafil at about 5 mg to about 20 mg orally twice daily.

46. The method of any one of claims 1, 4, 6, 17, 21, 25, and 31 wherein the method comprises administration of intravenous H2G that is followed by intranasal administration of a combination H2G/tenofovir spray twice daily.

47. A method for treating a disease or condition selected from the group consisting of acute infectious mononucleosis, chronic mononucleosis, long COVID, chronic fatigue syndrome, fibromyalgia, Crohn's disease, ulcerative colitis, rheumatoid arthritis, systemic lupus erythematosus, Graves' disease, Alzheimer's disease, mesial temporal lobe seizures, Epstein-Barr-virus-linked autism, and an Epstein-Barr-virus-linked cancer, the method comprising the steps of:
(a) administering H2G intravenously or intramuscularly at about 5 mg/kg to about 25 mg/kg twice daily or administering valomaciclovir stearate polymorph Form A orally at a high dose of about 1.5 g to 2 g orally twice daily in combination with administering an additional agent selected from the group consisting of: (i) artesunate administered intravenously at about 2.4 mg/kg twice daily for 1 day to 6 weeks; (ii) ribavirin administered orally or by inhalation at about 100 mg to about 500 mg twice daily for 1 day to 6 weeks; and (iii) mycophenolate administered orally or intravenously at about 500 mg to about 1.5 mg twice daily for 1 day to 6 weeks;
(b) subsequently administering low-dose valomaciclovir stearate polymorph Form A at a dosage of about 1 g to 2 g once daily with chronic dosing; and
(c) then subsequently administering a therapeutically effective quantity of an additional agent selected from the group consisting of an anti-CD20 agent, an anti-integrin α4β1 agent, and an anti-CD37 agent.

48. The method of claim 47 wherein the additional agent in step (a) is artesunate.

49. The method of claim 47 wherein the additional agent in step (a) is ribavirin.

50. The method of claim 47 wherein the additional agent in step (a) is mycophenolate.

51. The method of claim 47 wherein the agent administered in step (c) is an anti-integrin α4β1 agent, and wherein the anti-integrin α4β1 agent is natalizumab.

52. The method of claim 47 wherein the agent administered in step (c) is an anti-CD20 agent, and wherein the anti-CD20 agent is selected from the group consisting of rituximab, ocrelizumab, obinutuzumab, ofatumumab, ibritumomab tiuxetan, tositumomab, and ublituximab.

53. The method of claim 47 wherein the agent administered in step (c) is an anti-CD37 agent, and wherein the anti-CD37 agent is selected from the group consisting of lilotomab satetrextan, Humalutin, naratuximab emtansine, and otlertuzumab.

54. The method of claim 47 wherein the disease or condition is an Epstein-Barr virus-linked cancer.

55. The method of claim 54 wherein the Epstein-Barr virus-linked cancer is selected from the group consisting of nasopharyngeal carcinoma, glioblastoma multiforme, and Kaposi's sarcoma.

56. A method for treatment of glioblastoma comprising the steps of:
(a) administering an induction agent selected from the group consisting of:
(1) ribavirin administered at about 100 mg to about 400 mg twice daily for 1 day to 4 weeks;
(2) romidepsin administered at about 14 mg/m² on days 1 and 8; and
(3) sodium phenylbutyrate administered at a loading dose of about 250 mg intravenously over 90 minutes, followed by a dose of about 250 mg/kg per day for 7 days; followed by
(b) administering H2G intramuscularly or intravenously at a dose of about 5 mg/kg to about 25 mg/kg twice daily for 1 day to 6 weeks; followed by
(c) administering artesunate at a dose of about 1 mg/kg to about 2.4 mg/kg for 1 day to 6 weeks; followed by
(d) administering high dose polymorph Form A of valomaciclovir stearate at about 1.5 g to about 2 g twice daily for 1 week to 6 weeks, tenofovir disoproxil fumarate at about 75 mg twice daily for 1 day to 6 weeks, with the dose depending on the patient's renal function, and sildenafil at about 5 mg twice daily for 1 day to 6 weeks; and followed by
(e) administering about 850 mg of polymorph Form A of valomaciclovir stearate twice daily, tenofovir disoproxil fumarate at about 75 mg twice daily, with the dose and dosing duration depending on the patient's renal function with chronic dosing, and sildenafil at about 5 mg to about 20 mg with twice daily chronic dosing.

57. The method of claim 56 wherever the induction agent in step (a) is ribavirin.

58. The method of claim 56 wherever the induction agent in step (a) is romidepsin.

59. The method of claim 56 wherever the induction agent in step (a) is sodium phenylbutyrate.

60. A formulation of polymorph Form A of valomaciclovir stearate for oral administration, the formulation comprising, per unit dose:
(a) 1000 mg of the Form A polymorph of valomaciclovir stearate;
(b) 180.53 mg of croscarmellose sodium;
(c) 42.31 mg of Povidone K-30;
(d) 13.08 mg of polysorbate 80;
(e) 53.21 mg of talc; and
(f) 9.60 mg of magnesium stearate;
wherein the formulation is in the form of caplets.

61. A method for triple therapy of long COVID comprising the steps of:
(a) administering a therapeutically effective quantity of an induction agent for a human herpesvirus;
(b) administering a therapeutically effective quantity of at least one antiviral agent selected from the group consisting of H2G and polymorph Form A of valomaciclovir stearate, wherein the antiviral agent or agents are administered in two stages: a first high-dose stage and a second low-dose chronic stage; and
(c) administering a therapeutically effective quantity of an anti-B-cell agent selected from the group consisting of anti-CD20 agent, an anti-integrin α4β1 agent, or an anti-CD37 agent.

62. The method of claim 61 wherein the induction agent is selected from the group consisting of ribavirin, arginine, and an HDAC inhibitor.

63. The method of claim 62 wherein the induction agent is an HDAC inhibitor, and wherein the HDAC inhibitor is selected from the group consisting of valproic acid, sodium butyrate, romidepsin, abexinostat, belinostat, entinostat, givinostat, martinostat, mocetinostat, panobinostat, pracinostat, resminostat, sodium phenylbutyrate, tucidinostat, and vorinostat.

64. The method of claim 61 wherein the agent administered in step (c) is an anti-integrin α4β1 agent, and wherein the anti-integrin α4β1 agent is natalizumab.

65. The method of claim 61 wherein the agent administered in step (c) is an anti-CD20 agent, and wherein the anti-CD20 agent is selected from the group consisting of rituximab, ocrelizumab, obinutuzumab, ofatumumab, ibritumomab tiuxetan, tositumomab, and ublituximab.

66. The method of claim 61 wherein the agent administered in step (c) is an anti-CD37 agent, and wherein the anti-CD37 agent is selected from the group consisting of lilotomab satetrextan, Humalutin, naratuximab emtansine, and otlertuzumab.

67. The method of claim 61 wherein antiviral therapy is initiated at a high dose by administration of intravenous or intramuscular H2G (omaciclovir) at a dosage of about 5 mg/kg to about 25 mg/kg per day twice daily for 1 to 6 weeks.

68. The method of claim 61 wherein antiviral therapy is initiated at a high dose by administration of intravenous or intramuscular H2G (omaciclovir) at a dosage of about 5 mg/kg to about 25 mg/kg per day twice daily for 1 to 6 weeks.

69. The method of claim 61 wherein antiviral therapy is then continued at a low dose by administration of polymorph Form A of valomaciclovir stearate at about 500 mg to about 850 mg, tenofovir disoproxil fumarate at about 75 mg, and pharmaceutical grade cod liver oil at about 80 mg to about 500 mg twice daily with chronic dosing.

70. The method of claim 61 wherein antiviral therapy is then continued at a low dose by administration of polymorph Form A of valomaciclovir stearate at about 500 mg to about 850 mg, tenofovir disoproxil fumarate at about 75 mg, and sildenafil at about 5 mg to about 20 mg twice daily with chronic dosing.

71. A capsule or tablet formulation comprising:
(a) polymorph Form A of valomaciclovir stearate at about 500 mg to about 850 mg per capsule or tablet;
(b) tenofovir disoproxil fumarate at about 75 mg per capsule or table (with daily dosing of tenofovir disoproxil fumarate dependent on the patient's renal status); and
(c) pharmaceutical grade cod liver oil at about 80 mg to about 500 mg per capsule or tablet.

72. A capsule or tablet formulation comprising:
(a) polymorph Form A of valomaciclovir stearate at about 500 mg to about 850 mg per capsule or tablet;
(b) tenofovir disoproxil fumarate at about 75 mg per capsule or tablet (with daily dosing of tenofovir disoproxil fumarate dependent on the patient's renal status); and
(c) sildenafil at about 5 mg to about 20 mg per capsule or tablet.

73. A method for treating long COVID cases in which both persistent SARS-CoV-2 virus and reactivated Epstein-Barr virus or reactivated HHV6 virus is of concern, the method comprising the steps of:
(a) chronically administering a therapeutically effective quantity of nirmatrelvir;
(b) chronically administering a therapeutically effective quantity of ritonavir; and
(c) chronically administering a therapeutically effective quantity of polymorph Form A of valomaciclovir stearate.

74. The method of claim 73 wherein the method comprises the steps of:
(a) chronic administration of about 75 mg of nirmatrelvir twice daily;
(b) chronic administration of about 50 mg of ritonavir twice daily; and
(c) chronic administration of about 750 mg of polymorph Form A of valomaciclovir stearate twice daily.

75. A combination pill comprising:
(a) about 75 mg of nirmatrelvir per unit dose;
(b) about 50 mg of ritonavir per unit dose; and
(c) about 750 mg of polymorph Form A of valomaciclovir stearate per unit dose.

76. The method of any one of claims 1, 4, 6, 17, 21, 25, 31, 40, 47, 56, 61, and 73 wherein the method further comprises administration of a therapeutically effective quantity of testosterone.

77. The method of claim 76 wherein the testosterone is administered by a method or device selected from the group consisting of injections, patches, buccal patches, implants, transdermal films, gels, and transdermal solutions.

78. A method of treating long COVID comprising the step of administering a therapeutically effective quantity of a combination of therapeutic agents selected from the group consisting of:
(a) valomaciclovir stearate and remdesivir, wherein the remdesivir is administered orally; and
(b) H2G and remdesivir, wherein the remdesivir is administered intravenously.

79. The method of claim 78 wherein the valomaciclovir stearate is the polymorphic Form A of valomaciclovir stearate.

80. The method of claim 78 wherein remdesivir and valomaciclovir stearate are administered and wherein the active agents are administered in a dosage form that is pill, tablet, or capsule comprising about 300 mg valomaciclovir stearate plus about 700 mg remdesivir in each 1-gram tablet.

81. The method of claim 80 wherein the initial dose for long COVID is two 1-gram tablets twice daily for 4 days to 6 weeks followed by chronic dosing of one tablet once daily or twice daily.

82. The method of claim 80 wherein the dosage form is administered for 4 days to 6 weeks followed by once-daily dosing of either valomaciclovir stearate or a combination dosage form including valomaciclovir stearate, wherein the combination dosage form including valomaciclovir stearate is selected from: (i) a combination dosage form comprising a therapeutically effective quantity of valomaciclovir stearate and a therapeutically effective quantity of sildenafil; (ii) a combination dosage form comprising a therapeutically effective quantity of valomaciclovir stearate and a therapeutically effective quantity of tenofovir alafenamide fumarate; and (iii) a combination dosage form comprising a therapeutically effective quantity of valomaciclovir stearate and a therapeutically effective quantity of lysine.

83. A combination dosage form including valomaciclovir stearate, wherein the combination dosage form including valomaciclovir stearate is selected from: (i) a combination dosage form comprising a therapeutically effective quantity of valomaciclovir stearate and a therapeutically effective quantity of sildenafil; (ii) a combination dosage form comprising a therapeutically effective quantity of valomaciclovir stearate and a therapeutically effective quantity of tenofovir alafenamide fumarate; and (iii) a combination dosage form comprising a therapeutically effective quantity of valomaciclovir stearate and a therapeutically effective quantity of lysine.

84. The combination dosage form of claim 83 wherein the valomaciclovir stearate is the polymorphic Form A of valomaciclovir stearate.

85. The combination dosage form of claim 83 wherein the dosage form is formulated as a pill, a capsule, a tablet, a liquid, or a nasal spray.

86. A method of treating a disease or condition selected from the group consisting of long COVID, Alzheimer's disease, multiple sclerosis, chronic fatigue syndrome, fibromyalgia, acute infectious mononucleosis, and chronic mononucleosis comprising the step of administering a therapeutically effective quantity of valomaciclovir stearate and a therapeutically effective quantity of taurine.

87. The method of claim 86 wherein the valomaciclovir stearate is the polymorphic Form A of valomaciclovir stearate.

88. The method of claim 86 wherein the valomaciclovir stearate and taurine are administered in a capsule, pill, tablet, liquid, nasal spray, or patch.

89. A combination dosage form comprising a therapeutically effective quantity of valomaciclovir stearate and a therapeutically effective quantity of taurine.

90. The combination dosage form of claim 89 wherein the valomaciclovir stearate is the polymorphic Form A of valomaciclovir stearate.

91. The combination dosage form of claim 89 wherein the combination dosage form is formulated as a capsule, pill, tablet, liquid, nasal spray, or patch.
